(19) United States
(12) Patent Application Publication
(10) Pub. No.: US 2025/0352464 A1
(43) Pub. Date: Nov. 20, 2025

(54) TOPICAL COMPOSITIONS AND METHODS FOR TREATING DERMATOPOROSIS

(71) Applicant: GALDERMA HOLDING SA, ZUG (CH)

(72) Inventors: Alan WIDGEROW, Carlsbad, CA (US); John GARRUTO, Carlsbad, CA (US); Lucian IONESCU, Hawkesbury (CA)

(21) Appl. No.: 19/285,967

(22) Filed: Jul. 30, 2025

Related U.S. Application Data
(63) Continuation of application No. PCT/US2024/013522, filed on Jan. 30, 2024.
(60) Provisional application No. 63/442,424, filed on Jan. 31, 2023.

(51) Int. Cl. A61K 9/00; A61K 9/127; A61K 31/192; A61K 36/23; A61P 17/00

(52) U.S. Cl. CPC A61K 9/0014; A61K 9/127; A61K 31/192; A61K 36/23; A61P 17/00

(57) ABSTRACT

Disclosed herein are topical compositions comprising Centella asiatica extract and mandelic acid, wherein the compositions have a pH of less than 5, and methods of using the compositions to decrease cellular senescence in skin and treat dermatoporosis. Topical compositions comprising Centella asiatica extract and mandelic acid are effective to reduce the number of senescent cells in mammalian dermis, reduce the senescence-associated secretory phenotype (SASP), downregulate at least one SASP-associated gene, increase the expression of at least one cell cycle-related gene in dermal fibroblasts, downregulate at least one gene associated with skin inflammation, and increase fibroblast cell turnover.

### CROSS-REFERENCE TO RELATED APPLICATIONS

This application is a continuation of International PCT Application No. PCT/US2024/013522 filed Jan. 30, 2024, which application claims priority under 35 U.S.C. § 119(e) to U.S. Provisional Application No. 63/442,424, filed Jan. 31, 2023, the entire contents of which are incorporated herein by reference.

### BACKGROUND

The present disclosure relates generally to the field of topical compositions and methods of using the same. In particular, the present disclosure relates to topical compositions for treating and/or reducing the effects of dermatoporosis. Dermatoporosis is a term referring to chronic skin insufficiency and fragility that leads to a loss of function eventually resulting in a breakdown of the protective mechanisms of human skin. People with dermatoporosis exhibit atrophic, thinning skin that becomes fragile, exhibits purpura and white pseudoscars on the extremities, has a tendency to tear, and may lead to deep dissecting hematomas. See G. Kaya, et al., “Dermatoporosis, a Prevalent Skin Condition Affecting the Elderly: Current Situation and Potential Treatments,” 37 Clinics Dermatol. 346-50 (2019). Dermatoporosis progression may lead to skin lacerations, delayed healing, bleeding, skin infections, and eventually, to medical emergency.

Dermatoporosis first manifests at ages between 40 and 60 years, and the disease reaches full development at ages between 70 and 90 years—a potential 50-year span. Dermatoporosis is likely to increase in prevalence as those in the post-World War II “Baby Boom” cohort continue to age and the population shifts toward having a higher proportion of people aged 65 years or older. Among this cohort, fully-developed dermatoporosis is quite prevalent, with two French studies placing the prevalence at 32% and 37.5%. See J. H. Saurat, et al., “A Simple Self-Diagnosis Tool to Assess the Prevalence of Dermatoporosis in France,” 31 J. Eur. Acad. Dermatol. Venereol. 1380-86 (2017); V. Mengeaud, et al., “Prevalence of Dermatoporosis in Elderly French Hospital In-Patients: A Cross-Sectional Study,” 166 Br. J. Dermatol. 442-43 (2012). Thus, improved compositions and methods for treating and/or reducing the effects of dermatoporosis would present tremendous benefit.

### SUMMARY

In one aspect, which may be combined with any other aspect or embodiment, the present disclosure relates to a topical composition, comprising: Centella asiatica extract encapsulated within liposomes; and an alpha hydroxy acid (AHA), wherein the composition has a pH of less than 5. In some embodiments, the composition is in the form of an emulsion. In some embodiments, the emulsion is an oil-in-water (O/W) emulsion.

In some embodiments, the pH of the composition is greater than or equal to 4 and less than 5. In some embodiments, the Centella asiatica extract is present at a concentration of at least about 0.01 wt. % and less than 0.2 wt. %, relative to the total weight of the composition. In some embodiments, the Centella asiatica extract is present at a concentration of about 0.01 wt. % to about 0.1 wt. %, relative to the total weight of the composition.

In some embodiments, the Centella asiatica extract comprises triterpenes. In some embodiments, the triterpenes are one or more selected from the group consisting of: madecassoside, asiaticoside, madecassic acid, and asiatic acid. In some embodiments, the triterpenes are present in the Centella asiatica extract at a concentration of at least about 70 wt. %, relative to the total weight of the Centella asiatica extract. In some embodiments, the triterpenes are present in the composition at a concentration of at least about 0.005 wt. % and less than 0.2 wt. %, relative to the total weight of the composition. In some embodiments, triterpenes are present in the Centella asiatica extract at a concentration of at least about 0.01 wt. % to about 0.05 wt. %, relative to the total weight of the of the composition.

In some embodiments, the AHA comprises mandelic acid. In some embodiments, the AHA is present at a concentration of at least about 0.01 wt. % and less than 0.5 wt. %, relative to the total weight of the composition. In some embodiments, the AHA is present at a concentration of about 0.05 wt. % to about 0.3 wt. %, relative to the total weight of the composition.

In some embodiments, the liposomes are present at a concentration of about 0.03 wt. % to about 4 wt. %, relative to the total weight of the composition. In some embodiments, the liposomes comprise lecithin, propanediol, or a combination thereof. In some embodiments, the liposomes comprise an antioxidant. In some embodiments, the antioxidant is tocopherol, tocopheryl acetate, or a combination thereof. In some embodiments, the liposomes have a mean particle size of 100 nm to 300 nm. In some embodiments, the liposomes have a mean particle size of about 250 nm.

In some embodiments, the Centella asiatica extract is present in an active solution. In some embodiments, the liposomes and the active solution are present in a weight ratio of about 1:1 to about 1:20. In some embodiments, the liposomes and the active solution are present in a weight ratio of about 1:2 to about 1:10.

In some embodiments, the Centella asiatica extract and the AHA are present in a weight ratio of about 1:5 to 1:15.

In some embodiments, the topical composition further comprises an antioxidant separate from any antioxidant in the liposomes. In some embodiments, the antioxidant separate from any antioxidant in the liposomes is tocopheryl acetate, tocopherol, or a combination thereof.

In some embodiments, the topical composition further comprises at least one of a humectant, an emollient, or a humectant. In some embodiments, the topical composition further comprises glycerin.

In some embodiments, the topical composition is a cream or lotion.

In another aspect, which may be combined with any other aspect or embodiment, the present disclosure relates to a topical composition, comprising: Centella asiatica extract, wherein the Centella asiatica extract is encapsulated within liposomes; and an alpha hydroxy acid (AHA), wherein: the composition is an emulsion; the composition has a pH of 4 to 5; and the composition reduces biomarkers of skin inflammation in mammalian skin cells.

In another aspect, which may be combined with any other aspect or embodiment, the present disclosure relates to a topical composition, comprising: an alpha hydroxy acid (AHA); and triterpenes selected from the group consisting of: madecassoside, asiaticoside, madecassic acid, and asiatic acid, wherein the triterpenes are encapsulated within a liposome, and wherein the composition has a pH of 4 to 5. In some embodiments, the AHA comprises mandelic acid.

In some embodiments, the AHA is present in the composition at a concentration of at least about 0.01 wt. % and less than 0.5 wt. %, relative to the total weight of the composition.

In some embodiments, the triterpenes are present in the composition at a concentration of at least about 0.005 wt. % and less than 0.2 wt. %, relative to the total weight of the composition.

In some embodiments, the composition is an emulsion.

In some embodiments, a topical composition of the present disclosure is effective to reduce the number of senescent cells in mammalian dermis.

In some embodiments, a topical composition of the present disclosure is effective to reduce the senescence-associated secretory phenotype (SASP) in mammalian epidermal cells.

In some embodiments, a topical composition of the present disclosure is effective to downregulate at least one SASP-associated gene selected from the group consisting of IL8, IL1B, HIST1H2BG, and UBE2C.

In some embodiments, a topical composition of the present disclosure is effective to increase the expression of at least one cell cycle gene in mammalian dermal fibroblasts. In some embodiments, the at least one cell cycle gene in mammalian fibroblasts is selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH.

In some embodiments, a topical composition of the present disclosure is effective to downregulate at least one gene associate with skin inflammation selected from the group consisting of: MMP2, HSPA8, CSF2RA, CXCL1, IL2RB, NFKBIA, and PTGS2.

In some embodiments, a topical composition of the present disclosure is effective to downregulate at least one transcription factor of the AP-1 complex. In some embodiments, the transcription factor of the AP-1 complex is at least one selected from JunB and FosL2.

In some embodiments, a topical composition of the present disclosure is effective to activate cell cycle progression, by upregulating CCNE2, CCNB1, or a combination thereof.

In some embodiments, in a topical composition of the present disclosure, the Centella asiatica extract and the AHA are present at concentrations effective to synergistically downregulate at least one SASP-associated gene selected from the group consisting of IL8, IL1B, HIST1H2BG, and UBE2C.

In some embodiments, in a topical composition of the present disclosure, the Centella asiatica extract and the AHA are present at concentrations effective to synergistically upregulate at least one cell cycle gene in mammalian dermal fibroblasts selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH.

In some embodiments, in a topical composition of the present disclosure, the Centella asiatica extract and the AHA are present at concentrations effective to synergistically downregulate at least one SASP-associated gene selected from the group consisting of IL8, IL1B, HIST1H2BG, and UBE2C.

In some embodiments, in a topical composition of the present disclosure, the Centella asiatica extract and the AHA are present at concentrations effective to synergistically downregulate at least one transcription factor of the AP-1 complex. In some embodiments, the transcription factor of the AP-1 complex is at least one selected from JunB and FosL2.

In some embodiments, in a topical composition of the present disclosure, the Centella asiatica extract and the AHA are present at concentrations effective to synergistically activate cell cycle progression, by upregulating CCNE2, CCNB1, or a combination thereof.

In some embodiments, in a topical composition of the present disclosure, the triterpenes and the AHA are present at concentrations effective to synergistically downregulate at least one SASP-associated gene selected from the group consisting of IL8, IL1B, HIST1H2BG, and UBE2C.

In some embodiments, in a topical composition of the present disclosure, the triterpenes and the AHA are present at concentrations effective to synergistically upregulate at least one cell cycle gene in mammalian dermal fibroblasts selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH.

In some embodiments, in a topical composition of the present disclosure, the triterpenes and the AHA are present at concentrations effective to synergistically downregulate at least one SASP-associated gene selected from the group consisting of IL8, IL1B, HIST1H2BG, and UBE2C.

In some embodiments, in a topical composition of the present disclosure, the triterpenes and the AHA are present at concentrations effective to synergistically downregulate at least one transcription factor of the AP-1 complex. In some embodiments, the transcription factor of the AP-1 complex is at least one selected from JunB and FosL2.

In some embodiments, in a topical composition of the present disclosure, the triterpenes and the AHA are present at concentrations effective to synergistically activate cell cycle progression, by upregulating CCNE2, CCNB1, or a combination thereof.

In another aspect, which may be combined with any other aspect or embodiment, the present disclosure relates to a method of treating skin affected by dermatoporosis, the method comprising: administering a topical composition of the present disclosure to the affected skin.

In another aspect, which may be combined with any other aspect or embodiment, the present disclosure relates to a method of increasing fibroblast cell turnover in mammalian skin, the method comprising: administering the topical composition of any one of claims 1-47 to the mammalian skin.

In another aspect, which may be combined with any other aspect or embodiment, the present disclosure relates to a method of reducing the number of senescent cells in mammalian dermis, the method comprising: administering a topical composition of the present disclosure to mammalian skin.

In another aspect, which may be combined with any other aspect or embodiment, the present disclosure relates to a method of reducing the senescence-associated secretory phenotype (SASP) in mammalian epidermal cells, the method comprising: administering a topical composition of the present disclosure to mammalian skin.

In another aspect, which may be combined with any other aspect or embodiment, the present disclosure relates to a method of downregulating at least one SASP-associated gene in mammalian skin, the method comprising: administering a topical composition of the present disclosure to mammalian skin, wherein the at least one SASP-associated gene is selected from the group consisting of IL8, IL1B, HIST1H2BG, and UBE2C.

In another aspect, which may be combined with any other aspect or embodiment, the present disclosure relates to a method of increasing the expression of at least one cell cycle gene in mammalian dermal fibroblasts, the method comprising: administering a topical composition of the present disclosure to mammalian skin, wherein the at least one cell cycle gene in mammalian fibroblasts is selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH.

In another aspect, which may be combined with any other aspect or embodiment, the present disclosure relates to a method of downregulating at least one gene associated with skin inflammation, the method comprising: administering a topical composition of the present disclosure to mammalian skin, wherein the at least one gene associated with skin inflammation selected from the group consisting of: MMP2, HSPA8, CSF2RA, CXCL1, IL2RB, NFKBIA, and PTGS2.

In another aspect, which may be combined with any other aspect or embodiment, the present disclosure relates to a method of downregulating at least one transcription factor of the AP-1 complex, the method comprising: administering a topical composition of the present disclosure to mammalian skin. In some embodiments, the transcription factor of the AP-1 complex is at least one selected from JunB and FosL2.

In another aspect, which may be combined with any other aspect or embodiment, the present disclosure relates to a method of activating cell cycle progression, the method comprising: administering a topical composition of the present disclosure to mammalian skin, wherein the composition upregulates CCNE2, CCNB1, or a combination thereof.

Additional aspects and/or embodiments of the invention will be provided, without limitation, in the detailed description of the present technology set forth below. The following detailed description is exemplary and explanatory, but it is not intended to be limiting.

### BRIEF DESCRIPTION OF THE DRAWINGS

Various objects, aspects, features, and advantages of the disclosure will become more apparent and better understood by referring to the detailed description taken in conjunction with the accompanying figures.

FIG. 1A is a plot of fluorescence emission (590 nm) versus concentration of Centella asiatica extract for human dermal fibroblasts in an alamar blue assay.

FIG. 1B is a plot of fluorescence emission (590 nm) versus concentration of Centella asiatica extract for human dermal keratinocytes in an alamar blue assay.

FIG. 1C is a plot of fluorescence emission (590 nm) versus concentration of mandelic acid for human dermal fibroblasts in an alamar blue assay.

FIG. 1D is a plot of fluorescence emission (590 nm) versus concentration of mandelic acid for human dermal keratinocytes in an alamar blue assay.

FIG. 2 is a schematic illustration of biomarkers related to senescence in dermal cells.

FIG. 3A is a plot of IL8 gene expression in human keratinocytes grown to confluence and treated with compositions comprising Centella asiatica extract, mandelic acid, or a combination of Centella asiatica extract and mandelic acid.

FIG. 3B is a plot of IL1B gene expression in human keratinocytes grown to confluence and treated with compositions comprising Centella asiatica extract, mandelic acid, or a combination of Centella asiatica extract and mandelic acid.

FIG. 3C is a plot of HIST1H2BG gene expression in human keratinocytes grown to confluence and treated with compositions comprising Centella asiatica extract, mandelic acid, or a combination of Centella asiatica extract and mandelic acid.

FIG. 3D is a plot of UBE2C gene expression in human keratinocytes grown to confluence and treated with compositions comprising Centella asiatica extract, mandelic acid, or a combination of Centella asiatica extract and mandelic acid.

FIG. 4A is a plot of MMP2 gene expression (fold change) in human keratinocytes grown to confluence and treated with compositions comprising Centella asiatica extract, mandelic acid, or a combination of Centella asiatica extract and mandelic acid.

FIG. 4B is a plot of HSPA8 gene expression (fold change) in human keratinocytes grown to confluence and treated with compositions comprising Centella asiatica extract, mandelic acid, or a combination of Centella asiatica extract and mandelic acid.

FIG. 4C is a plot of CSF2RA gene expression (fold change) in human keratinocytes grown to confluence and treated with compositions comprising Centella asiatica extract, mandelic acid, or a combination of Centella asiatica extract and mandelic acid.

FIG. 4D is a plot of CXCL1 gene expression (fold change) in human keratinocytes grown to confluence and treated with compositions comprising Centella asiatica extract, mandelic acid, or a combination of Centella asiatica extract and mandelic acid.

FIG. 4E is a plot of IL2RB gene expression (fold change) in human keratinocytes grown to confluence and treated with compositions comprising Centella asiatica extract, mandelic acid, or a combination of Centella asiatica extract and mandelic acid.

FIG. 4F is a plot of NFKBIA gene expression (fold change) in human keratinocytes grown to confluence and treated with compositions comprising Centella asiatica extract, mandelic acid, or a combination of Centella asiatica extract and mandelic acid.

FIG. 4G is a plot of PTGS2 gene expression (fold change) in human keratinocytes grown to confluence and treated with compositions comprising Centella asiatica extract, mandelic acid, or a combination of Centella asiatica extract and mandelic acid.

FIG. 5 is a plot of CDKN2B gene expression (fold change) in human keratinocytes grown to confluence and treated with compositions comprising Centella asiatica extract, mandelic acid, or a combination of Centella asiatica extract and mandelic acid.

FIG. 6 is a plot of gene expression (fold change) for cell cycle-related genes upregulated in human dermal fibroblasts grown to confluence and treated with compositions comprising Centella asiatica extract, mandelic acid, or a combination of Centella asiatica extract and mandelic acid.

FIG. 7A is a plot of WEE1 gene expression (fold change) in human dermal fibroblasts grown to confluence and treated with compositions comprising Centella asiatica extract, mandelic acid, or a combination of Centella asiatica extract and mandelic acid.

FIG. 7B is a plot of H2AFX gene expression (fold change) in human dermal fibroblasts grown to confluence and treated with compositions comprising Centella asiatica extract, mandelic acid, or a combination of Centella asiatica extract and mandelic acid.

FIG. 7C is a plot of LMNB1 gene expression (fold change) in human dermal fibroblasts grown to confluence and treated with compositions comprising Centella asiatica extract, mandelic acid, or a combination of Centella asiatica extract and mandelic acid.

FIG. 7D is a plot of CCNB2 gene expression (fold change) in human dermal fibroblasts grown to confluence and treated with compositions comprising Centella asiatica extract, mandelic acid, or a combination of Centella asiatica extract and mandelic acid.

FIG. 7E is a plot of BUB1 gene expression (fold change) in human dermal fibroblasts grown to confluence and treated with compositions comprising Centella asiatica extract, mandelic acid, or a combination of Centella asiatica extract and mandelic acid.

FIG. 7F is a plot of CDC45 gene expression (fold change) in human dermal fibroblasts grown to confluence and treated with compositions comprising Centella asiatica extract, mandelic acid, or a combination of Centella asiatica extract and mandelic acid.

FIG. 7G is a plot of CDK1 gene expression (fold change) in human dermal fibroblasts grown to confluence and treated with compositions comprising Centella asiatica extract, mandelic acid, or a combination of Centella asiatica extract and mandelic acid.

FIG. 7H is a plot of BIRC5 gene expression (fold change) in human dermal fibroblasts grown to confluence and treated with compositions comprising Centella asiatica extract, mandelic acid, or a combination of Centella asiatica extract and mandelic acid.

FIG. 7I is a plot of MCM10 gene expression (fold change) in human dermal fibroblasts grown to confluence and treated with compositions comprising Centella asiatica extract, mandelic acid, or a combination of Centella asiatica extract and mandelic acid.

FIG. 7J is a plot of CCNE2 gene expression (fold change) in human dermal fibroblasts grown to confluence and treated with compositions comprising Centella asiatica extract, mandelic acid, or a combination of Centella asiatica extract and mandelic acid.

FIG. 8 is a plot of gene expression (fold-change versus non-treated group) for JunB and related proteins in human dermal fibroblasts grown to confluence and treated with compositions comprising a combination of Centella asiatica extract and mandelic acid.

FIG. 9A shows p16-stained (yellow) human skin samples that were treated with a topical composition according to the present disclosure, compared to an untreated cell sample. Nuclei are in blue. The circled regions show the presence of senescent cells in the dermis (untreated) and movement of senescent cells into the epidermis (test composition).

FIG. 9B shows p16-stained (yellow) human skin samples that were treated with a topical composition according to the present disclosure, compared to an untreated cell sample. Nuclei are in blue. The circled regions show the presence of senescent cells in the dermis (untreated) and movement of senescent cells into the epidermis (test composition). Senescent cells in the center of the image for the untreated sample are present in a hair follicle, where cells undergo natural senescence as part of the hair cycle.

FIG. 9C shows p16-stained (yellow) human skin samples that were treated with a topical composition according to the present disclosure, compared to an untreated cell sample. Nuclei are in blue. The circled regions show the presence of senescent cells in the dermis (untreated) and movement of senescent cells into the epidermis (test composition). Senescent cells in the right-hand side of the image for the untreated sample and in the lower-left to center of the image for the treated sample are present in hair follicles, where cells undergo natural senescence as part of the hair cycle.

FIG. 9D shows p16-stained (yellow) human skin samples that were treated with a topical composition according to the present disclosure, compared to an untreated cell sample. Nuclei are in blue. The circled regions show the presence of senescent cells in the dermis (untreated) and movement of senescent cells into the epidermis (test composition). Senescent cells in the bottom of the image for the untreated sample and in the middle to upper portion of the image for the treated sample are present in hair follicles, where cells undergo natural senescence as part of the hair cycle.

### DETAILED DESCRIPTION

In the following description, numerous specific details are set forth in order to provide a thorough understanding of the present technology. Particular exemplary embodiments of the present technology may be implemented without some or all of these specific details. In other instances, well known process operations have not been described in detail in order not to unnecessarily obscure the present technologies.

### Topical Compositions

Centella asiatica Extract

Topical compositions according to the present disclosure comprise Centella asiatica extract. Centella asiatica is a traditional medicinal herb widely used in Asia and which is becoming more widely used in Western nations. Centella asiatica extracts are used as natural remedies with a broad range of uses in wound healing, skin barrier reinforcement, water retention in skin, reducing skin wrinkles, and mitigating skin conditions such as acne, atopic dermatitis, and burns. Its use in wound healing is attributed to its activity in improving collagen synthesis and microcirculatory function. It is also thought to regulate cellular senescence and to possess anti-hyaluronidase activity. See generally, e.g., E. Arribas-Lopez, et al., “A Systematic Review of the Effect of Centella Asiatica on Wound Healing,” 19 Int'l J. Envtl. Res. Pub. Health 3266 (2022); S. Saeidinia, et al., “Partial Thickness Burn Wound Healing by Topical Treatment: A Randomized Controlled Comparison Between Silver Sulfadiazine and Centiderm,” 96 Medicine 103 (2017); N. K. Nema, et al. “Matrix Metalloproteinase, Hyaluronidase and Elastase Inhibitory Potential of Standardized Extract of Centella asiatica,” 51 Pharm. Biol. 1182-87 (2013); Y. J. Kim, et al., “Centella asiatica Extracts Modulate Hydrogen Peroxide-Induced Senescence in Human Dermal Fibroblasts,” 20 Exptl. Dermatol. 998-1003 (2011).

Centella asiatica extract is available as an aqueous or organic extract, the therapeutic properties of which are attributed to the presence of triterpenes (triterpenes): madecassoside, asiaticoside, madecassic acid, and asiatic acid. Centella asiatica extracts may comprise high concentrations of triterpenes. For instance, commercially available Centella asiatica extracts (e.g., HETEROSIDES, available from Seppic) may contain greater than 70 wt. % triterpenes. In some embodiments the Centella asiatica extract comprises HETEROSIDES (Seppic). In some embodiments, the Centella asiatica extract comprises triterpenes selected from the group consisting of: madecassoside, asiaticoside, madecassic acid, and asiatic acid.

The Centella asiatica extract comprises triterpenes at any suitable concentration for achieving a therapeutic benefit (e.g., decreasing cellular senescence). In some embodiments, the triterpenes are present in the Centella asiatica extract at a concentration, relative to the total weight of the Centella asiatica extract, of greater than or equal to about 5 wt. %, greater than or equal to about 8 wt. %, greater than or equal to about 10 wt. %, greater than or equal to about 15 wt. %, greater than or equal to about 20 wt. %, greater than or equal to about 25 wt. %, greater than or equal to about 30 wt. %, greater than or equal to about 35 wt. %, greater than or equal to about 40 wt. %, greater than or equal to about 45 wt. %, greater than or equal to about 50 wt. %, greater than or equal to about 55 wt. %, greater than or equal to about 60 wt. %, greater than or equal to about 65 wt. %, greater than or equal to about 70 wt. %, greater than or equal to about 75 wt. %, greater than or equal to about 80 wt. %, greater than or equal to about 85 wt. %, greater than or equal to about 90 wt. %, greater than or equal to about 95 wt. %, or any range or value therein. In some embodiments, the Centella asiatica extract comprises triterpenes at a concentration of greater than or equal to 70 wt. %, relative to the total weight of the Centella asiatica extract.

The Centella asiatica extract (including triterpenes and any solvents, including water) is present in the topical composition at any suitable concentration suitable for achieving a therapeutic benefit (e.g., decreasing cellular senescence). In some embodiments, the Centella asiatica extract is present in the topical composition at a concentration, relative to the total weight of the topical composition, of at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.15 wt. %, at least about 0.20 wt. %, at least about 0.25 wt. %, at least about 0.30 wt. %, at least about 0.35 wt. %, at least about 0.40 wt. %, at least about 0.45 wt. %, at least about 0.50 wt. %, or any range or value therein between.

In some embodiments, the Centella asiatica extract is present in the topical composition at a concentration, relative to the total weight of the topical composition, of less than or equal to about 0.5 wt. %, less than or equal to about 0.45 wt. %, less than or equal to about 0.40 wt. %, less than or equal to about 0.35 wt. %, less than or equal to about 0.30 wt. %, less than or equal to about 0.25 wt. %, less than or equal to about 0.20 wt. %, less than or equal to about 0.15 wt. %, less than or equal to about 0.10 wt. %, less than or equal to about 0.09 wt. %, less than or equal to about 0.08 wt. %, less than or equal to about 0.07 wt. %, less than or equal to about 0.06 wt. %, less than or equal to about 0.05 wt. %, or any range or value therein between.

In some embodiments, the Centella asiatica extract is present in the topical composition at a concentration, relative to the total weight of the topical composition, of at least about 0.01 wt. % and less than 0.2 wt. %, about 0.01 wt. % to about 0.15 wt. %, about 0.01 wt. % to about 0.10 wt. %, about 0.01 wt. % to about 0.08 wt. %, about 0.01 wt. % to about 0.05 wt. %, or any range or value therein.

The embodiments provided above include illustrative concentration amounts for Centella asiatica extract based on high concentrations of triterpenes (e.g., greater than 70% triterpenes available from Seppic). In those instances where lower triterpene amounts are present in the Centella asiatica extract, the Centella asiatica extract concentrations of the illustrative examples can be increased to ensure that a minimal amount of triterpene concentration as disclosed herein is present in the formulation.

In some embodiments, the Centella asiatica extract is present in an active solution. As used herein, the term “active solution” refers to a solution containing the Centella asiatica extract (including any terpenes) and a solvent. In some embodiments, the Centella asiatica extract is present in the composition at concentrations lower than recommended for formulating Centella asiatica extract into topical compositions (e.g., recommended about 0.2 wt. % to 0.5 wt. %). Thus, in some embodiments, the Centella asiatica extract is present as an active solution at a concentration of 0.01 wt. % to less than 0.2 wt. % (e.g., 0.01 wt. % to 0.15 wt. %, 0.01 wt. % to 0.10 wt. %, 0.01 wt. % to 0.08 wt. %, 0.01 wt. % to 0.05 wt. %), relative to the total weight of the composition. The present inventors surprisingly discovered that low concentrations (e.g., less than 0.2 wt. %) of Centella asiatica extract afford numerous therapeutic benefits, discussed below (see Methods of Use). In some embodiments, low concentrations of Centella asiatica extract achieve synergistic therapeutic activity with low concentrations of alpha hydroxy acids (AHAs).

Given the range of concentrations of the Centella asiatica extract (e.g., at least about 0.01 wt. % and less than 0.2 wt. %) in the total composition and the concentration of triterpenes in the Centella asiatica extract (e.g., at least about 5 wt. % to about 95 wt. %), relative to the total weight of the Centella asiatica extract, it is evident that triterpenes may be present, with or without being part of a Centella asiatica extract, at a suitable concentration to achieve a therapeutic benefit (e.g., in reducing cellular senescence). In some embodiments, triterpenes may be present in the topical composition at a concentration, by weight relative to the total weight of the topical composition, of at least about 0.0005 wt. %, at least about 0.001 wt. %, at least about 0.002 wt. %, at least about 0.003 wt. %, at least about 0.004 wt. %, at least about 0.005 wt. %, at least about 0.006 wt. %, at least about 0.007 wt. %, at least about 0.008 wt. %, at least about 0.009 wt. %, at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.11 wt. %, at least about 0.12 wt. %, at least about 0.13 wt. %, at least about 0.14 wt. %, at least about 0.15 wt. %, at least about 0.16 wt. %, at least about 0.17 wt. %, at least about 0.18 wt. %, at least about 0.19 wt. %, or any range of value therein between.

In some embodiments, triterpenes may be present in the topical composition at a concentration, by weight relative to the total weight of the topical composition, of less than about 0.2 wt. %, less than or equal to about 0.19 wt. %, less than or equal to about 0.18 wt. %, less than or equal to about 0.17 wt. %, less than or equal to about 0.16 wt. %, less than or equal to about 0.15 wt. %, less than or equal to about 0.14 wt. %, less than or equal to about 0.13 wt. %, less than or equal to about 0.12 wt. %, less than or equal to about 0.11 wt. %, less than or equal to about 0.10 wt. %, less than or equal to about 0.09 wt. %, less than or equal to about 0.08 wt. %, less than or equal to about 0.09 wt. %, less than or equal to about 0.08 wt. %, less than or equal to about 0.07 wt. %, less than or equal to about 0.06 wt. %, less than or equal to about 0.05 wt. %, less than or equal to about 0.04 wt. %, less than or equal to about 0.05 wt. %, less than or equal to about 0.04 wt. %, less than or equal to about 0.03 wt. %, less than or equal to about 0.02 wt. %, less than or equal to about 0.01 wt. %, or any range or value therein between.

In some embodiments, triterpenes may be present in the topical composition at a concentration, by weight relative to the total weight of the topical composition, of at least about 0.0005 wt. % and less than 0.2 wt. %, 0.001 wt. % and less than 0.2 wt. %, about 0.001 wt. % to about 0.15 wt. %, about 0.001 wt. % to about 0.1 wt. %, about 0.001 wt. % to about 0.05 wt. %, about 0.001 wt. % to about 0.01 wt. %, at least about 0.005 wt. % and less than 0.2 wt. %, about 0.005 wt. % to about 0.15 wt. %, about 0.005 wt. % to about 0.10 wt. %, about 0.005 wt. % to about 0.05 wt. %, about 0.005 wt. %, to about 0.01 wt. %, or any range or value therein.

### Liposomes

The triterpenes present in Centella asiatica extract have high molecular weights and are chemically unstable, inhibiting skin adsorption after topical administration. Thus, in some embodiments, the Centella asiatica extract (and the triterpenes therein) are encapsulated within a delivery vehicle (e.g., liposomes, lipid nanoparticles, or a combination thereof) to improve stability of triterpenes and improve delivery of Centella asiatica extract into the skin by improving penetration into the epidermis and dermis. In some embodiments, the Centella asiatica extract is encapsulated within liposomes. Liposome compositions may improve distribution, efficacy, bioavailability, and/or activity of the active ingredient by improving delivery and tissue (e.g., skin) penetration. In some instances, improved delivery and skin penetration result from the active ingredient being encapsulated within a liposome.

Lecithin and other phospholipids may be used to prepare liposomes encapsulating the Centella asiatica extract. Formation of liposomes occurs when phospholipids such as lecithin are placed in water and consequently form one bilayer or a series of bilayers, each separated by water molecules, once enough energy is supplied. Liposomes may be formed by sonicating phospholipids in water. Low shear rates create multilamellar liposomes. Continued high-shear sonication tends to form smaller unilamellar liposomes. Hydrophobic compounds (e.g., triterpenes, antioxidants such as tocopherol and/or tocopheryl acetate, and oils such as sunflower seed oil) can be dissolved into the phospholipid bilayer membrane. The lipid bilayers of the liposomes deliver the Centella asiatica extract as described herein.

The phospholipids used to prepare the liposomes described herein may have a transition phase temperature of about 10° C. to about 25° C. In some embodiments, the phospholipids have a transition phase temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some embodiments, the phospholipids have a transition phase temperature in a range of about 10° C. to about 40° C., about 12° C. to about 36° C., about 14° C. to about 32° C., about 16° C. to about 20° C., or about 21° C. to about 25° C.

In some embodiments a liposome encapsulating Centella asiatica extract is prepared by a method comprising: combining an “active solution” which is a solution containing the Centella asiatica extract and a solvent (e.g., water) to form a mixture; and contacting the active solution with an aqueous solution comprising liposomes. In some instances, the contacting occurs at a temperature between about 10° C. and about 25° C. In some instances, the contacting occurs at a temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the contacting occurs at a temperature in a range of about 10° C. to about 40° C., about 12° C. to about 36° C., about 14° C. to about 32° C., about 16° C. to about 20° C., or about 21° C. to about 25° C.

In some embodiments, the solvent is water. In some embodiments, the solvent is an organic solvent. Exemplary organic solvents include, but are not limited to, petroleum ether, cyclohexane, toluene, carbon tetrachloride, dichloromethane, chloroform, diethyl ether, diisopropyl ether, ethyl acetate, butanol, n-propanol, ethanol, methanol, polyethylene glycol, propylene glycol, and pyridine. In some instances, the solvent is a glycol. In some instances, the solvent is butylene glycol. In some instances, the solvent is caprylyl glycol. In some instances, the solvent is propanediol (propylene glycol).

The solvent may be used at any suitable concentration for forming stable liposomes. In some embodiments, the solvent (e.g., propanediol) is provided at a concentration, by weight relative to the total weight of the liposomes, of greater than or equal to about 0.001 wt. %, greater than or equal to about 0.005 wt. %, greater than or equal to about 0.01 wt. %, greater than or equal to about 0.02 wt. %, greater than or equal to about 0.05 wt. %, greater than or equal to about 0.10 wt. %, greater than or equal to about 0.20 wt. %, greater than or equal to about 0.25 wt. %, greater than or equal to about 0.50 wt. %, greater than or equal to about 0.75 wt. %, greater than or equal to about 1.0 wt. %, greater than or equal to about 1.5 wt. %, greater than or equal to about 2.0 wt. %, greater than or equal to about 2.5 wt. %, greater than or equal to about 3.0 wt. %, greater than or equal to about 3.5 wt. %, greater than or equal to about 4.0 wt. %, greater than or equal to about 4.5 wt. %, greater than or equal to about 5.0 wt. %, greater than or equal to about 5.5 wt. %, greater than or equal to about 6.0 wt. %, greater than or equal to about 6.5 wt. %, greater than or equal to about 7.0 wt. %, greater than or equal to about 8 wt. %, greater than or equal to about 9 wt. %, greater than or equal to about 10 wt. %, or any range or value therein between In some embodiments, the solvent is selected from propanediol, butylene glycol, caprylyl glycol, or combinations thereof. In some embodiments, the solvent is propanediol.

In some embodiments, forming the liposomes encapsulating Centella asiatica extract comprises combining the Centella asiatica extract and a solvent (e.g., water) to form an active solution; and contacting the active solution with an aqueous solution comprising liposomes, wherein the aqueous solution comprises a water and liposomes at any suitable concentration for forming stable liposomes and encapsulating the Centella asiatica extract in the liposomes. In some embodiments, the aqueous solution comprises water at greater than or equal to about 20 wt. %, 30 wt. %, 40 wt. %, 50 wt. %, 60 wt. %, 70 wt. %, 80 wt. %, 90 wt. %, relative to the total weight of the liposomes, water, and Centella asiatica extract. In some embodiments, the aqueous solution comprises water in a range of about 10 wt. % to about 95 wt. %, about 20 wt. % to about 90 wt. %, about 30 wt. % to about 85 wt. %, about 40 wt. % to about 80 wt. %, or about 50 wt. % to about 60 wt. %, relative to the total weight of the liposomes, water, and Centella asiatica extract.

In some embodiments, the aqueous solution comprises liposomes at a concentration, relative to the total weight of the liposomes, water, and Centella asiatica extract, of at least or about 10 wt. %, at least about 20 wt. %, at least about 30 wt. %, at least about 40 wt. %, at least about 50 wt. %, at least about 60 wt. %, or any range or value therein between. In some embodiments, the aqueous solution comprises liposomes at a concentration, relative to the total weight of the liposomes, water, and Centella asiatica extract, of about 10 wt. % to about 80 wt. %, about 20 wt. % to about 70 wt. %, or about 30 wt. % to about 60 wt. %. A ratio of liposomes to water (w/w) may be in a range of about 1:9 to about 3:7. In some embodiments, the ratio of liposomes to water (w/w) may be greater than or equal to about 1:10, greater than or equal to about 1:9, greater than or equal to about 1:8, greater than or equal to about 1:7, greater than or equal to about 1:6, greater than or equal to about 1:5, greater than or equal to about 1:4, greater than or equal to about 1:3, or greater than or equal to about 1:2.

Methods for generation of liposomes encapsulating Centella asiatica extract may result in an entrapment efficiency of no more than 100%. In some embodiments, the entrapment efficiency is no more than 50%, 60%, 70%, 80%, 90%, 95%, 99%, or 99.5%.

The liposomes may be present in the topical composition at any suitable concentration to improve delivery of Centella asiatica extract into the extracellular matrix of the epidermal or dermal layers of the skin. In some embodiments, the liposomes are present in the topical composition at a concentration, by weight relative to the total weight of the composition, of greater than or equal to about 0.01 wt. %, greater than or equal to about 0.02 wt. %, greater than or equal to about 0.03 wt. %, greater than or equal to about 0.04 wt. %, greater than or equal to about 0.05 wt. %, greater than or equal to about 0.06 wt. %, greater than or equal to about 0.07 wt. %, greater than or equal to about 0.08 wt. %, greater than or equal to about 0.09 wt. %, greater than or equal to about 0.10 wt. %, greater than or equal to about 0.15 wt. %, greater than or equal to about 0.20 wt. %, greater than or equal to about 0.25 wt. %, greater than or equal to about 0.30 wt. %, greater than or equal to about 0.35 wt. %, greater than or equal to about 0.40 wt. %, greater than or equal to about 0.50 wt. %, greater than or equal to about 0.6 wt. %, greater than or equal to about 0.7 wt. %, greater than or equal to about 0.8 wt. %, greater than or equal to about 0.9 wt. %, greater than or equal to about 1.0 wt. %, greater than or equal to about 1.5 wt. %, greater than or equal to about 2.0 wt. %, greater than or equal to about 2.5 wt. %, greater than or equal to about 3.0 wt. %, greater than or equal to about 3.5 wt. %, greater than or equal to about 4.0 wt. %, greater than or equal to about 4.5 wt. %, greater than or equal to about 5.0 wt. %, or any range or value therein between.

In some embodiments, the liposomes are present in the topical composition at a concentration, by weight relative to the total weight of the composition, of about 0.01 wt. % to about 5 wt. %, about 0.05 wt. % to about 5 wt. %, about 0.1 wt. % to about 5 wt. %, about 0.03 wt. % to about 4 wt. %, about 0.05 wt. % to about 4 wt. %, about 0.05 wt. % to about 3 wt. %, about 0.05 wt. % to about 2 wt. %, about 0.05 wt. % to about 1 wt. %, about 0.05 wt. % to about 0.5 wt. %, about 0.05 wt. % to about 0.1 wt. %, about 0.1 wt. % to about 2 wt. %, about 0.1 wt. % to about 1 wt. %, about 0.2 wt. % to about 0.8 wt. %, about 0.3 wt. % to about 0.7 wt. %, or any range or value therein between.

In some embodiments, the liposomes have any suitable size to improve delivery of Centella asiatica extract into the skin. In some embodiments, the liposomes have an average diameter of greater than or equal to about 50 nm, greater than or equal to about 60 nm, greater than or equal to about 70 nm, greater than or equal to about 80 nm, greater than or equal to about 90 nm, greater than or equal to about 100 nm, greater than or equal to about 110 nm, greater than or equal to about 120 nm, greater than or equal to about 130 nm, greater than or equal to about 140 nm, greater than or equal to about 150 nm, greater than or equal to about 160 nm, greater than or equal to about 170 nm, greater than or equal to about 180 nm, greater than or equal to about 190 nm, greater than or equal to about 200 nm, greater than or equal to about 210 nm, greater than or equal to about 220 nm, greater than or equal to about 230 nm, greater than or equal to about 240 nm, greater than or equal to about 250 nm, greater than or equal to about 260 nm, greater than or equal to about 270 nm, greater than or equal to about 280 nm, greater than or equal to about 290 nm, greater than or equal to about 300 nm, greater than or equal to about 310 nm, greater than or equal to about 320 nm, greater than or equal to about 330 nm, greater than or equal to about 340 nm, greater than or equal to about 350 nm, greater than or equal to about 360 nm, greater than or equal to about 370 nm, greater than or equal to about 380 nm, greater than or equal to about 390 nm, greater than or equal to about 400 nm, greater than or equal to about 410 nm, greater than or equal to about 420 nm, greater than or equal to about 430 nm, greater than or equal to about 440 nm, greater than or equal to about 450 nm, greater than or equal to about 500 nm, greater than or equal to about 500 nm, or any range or value therein between.

In some embodiments, the liposomes have an average diameter of about 50 nm to about 500 nm, about 50 nm to about 400 nm, about 50 nm to about 300 nm, about 50 nm to about 250 nm, about 100 nm to about 500 nm, about 100 nm to about 400 nm, about 100 nm to about 300 nm, 100 nm to about 250 nm, about 200 nm to about 500 nm, about 200 nm to about 400 nm, about 200 nm to about 300 nm, or any range or value therein. In some embodiments, the liposomes have an average diameter of about 250 nm.

In some embodiments, the liposomes have a polydispersity index (PdI) of 0 to about 0.2. In some embodiments, the polydispersity index is about 0.01, 0.025, 0.05, 0.1, 0.25, 0.3, 0.35, 0.4, 0.45, 0.5, 0.55, 0.6, 0.65, 0.7, 0.75, or 0.8. In some instances, the polydispersity index is in a range of about 0.01 to about 0.8, about 0.025 to about 0.75, about 0.05 to about 0.6, or about 0.1 to about 0.3.

In some embodiments, the liposomes comprise propanediol, lecithin, or a combination thereof. In some embodiments, the propanediol is present in the liposomes at a concentration, by weight relative to the total weight of the liposomes, of greater than or equal to about 0.001 wt. %, greater than or equal to about 0.005 wt. %, greater than or equal to about 0.01 wt. %, greater than or equal to about 0.02 wt. %, greater than or equal to about 0.05 wt. %, greater than or equal to about 0.10 wt. %, greater than or equal to about 0.20 wt. %, greater than or equal to about 0.25 wt. %, greater than or equal to about 0.50 wt. %, greater than or equal to about 0.75 wt. %, greater than or equal to about 1.0 wt. %, greater than or equal to about 1.5 wt. %, greater than or equal to about 2.0 wt. %, greater than or equal to about 2.5 wt. %, greater than or equal to about 3.0 wt. %, greater than or equal to about 3.5 wt. %, greater than or equal to about 4.0 wt. %, greater than or equal to about 4.5 wt. %, greater than or equal to about 5.0 wt. %, greater than or equal to about 5.5 wt. %, greater than or equal to about 6.0 wt. %, greater than or equal to about 6.5 wt. %, greater than or equal to about 7.0 wt. %, greater than or equal to about 8 wt. %, greater than or equal to about 9 wt. %, greater than or equal to about 10 wt. %, or any range or value therein between.

In some embodiments, the propanediol is present in the liposomes at a concentration, by weight relative to the total weight of the liposomes or relative to the total weight of the topical composition, of about 0.001 wt. % to about 6 wt. %, about 0.002 wt. % to about 4 wt. %, about 0.01 wt. % to about 3 wt. %, or about 0.02 wt. % to about 2 wt. % by weight.

In some embodiments, the lecithin is present in the liposomes at a concentration, by weight relative to the total weight of the liposomes, of greater than or equal to about 0.001 wt. %, greater than or equal to about 0.005 wt. %, greater than or equal to about 0.01 wt. %, greater than or equal to about 0.02 wt. %, greater than or equal to about 0.05 wt. %, greater than or equal to about 0.10 wt. %, greater than or equal to about 0.20 wt. %, greater than or equal to about 0.25 wt. %, greater than or equal to about 0.50 wt. %, greater than or equal to about 0.75 wt. %, greater than or equal to about 1.0 wt. %, greater than or equal to about 1.5 wt. %, greater than or equal to about 2.0 wt. %, greater than or equal to about 2.5 wt. %, greater than or equal to about 3.0 wt. %, greater than or equal to about 3.5 wt. %, greater than or equal to about 4.0 wt. %, greater than or equal to about 4.5 wt. %, greater than or equal to about 5.0 wt. %, greater than or equal to about 5.5 wt. %, greater than or equal to about 6.0 wt. %, greater than or equal to about 6.5 wt. %, greater than or equal to about 7.0 wt. %, greater than or equal to about 8 wt. %, greater than or equal to about 9 wt. %, greater than or equal to about 10 wt. %, or any range or value therein between.

In some embodiments, the lecithin is present in the liposomes at a concentration, by weight relative to the total weight of the liposomes or relative to the total weight of the topical composition, of about 0.001 wt. % to about 6 wt. %, about 0.002 wt. % to about 4 wt. %, about 0.01 wt. % to about 3 wt. %, or about 0.02 wt. % to about 2 wt. % by weight.

In some embodiments, the liposomes are commercially available (e.g., PRO-LIPO™ NEO). In some embodiments, the liposomes comprise propanediol, lecithin, sunflower seed oil, and tocopherol.

### Alpha Hydroxy Acids (AHA)

Topical compositions according to the present disclosure comprise one or more alpha hydroxy acids (AHAs). In some embodiments, the one or more AHAs is selected from the group consisting of: mandelic acid, glycolic acid, lactic acid, citric acid, malic acid, hydroxycaprylic acid, hydroxycapric acid. In some embodiments, the one or more AHAs comprises mandelic acid. In some embodiments, the mandelic acid comprises (S)-mandelic acid. In some embodiments the mandelic acid comprises (R)-mandelic acid. In some embodiments, the mandelic acid comprises (S)-mandelic acid and (R)-mandelic acid (e.g., paramandelic acid).

Mandelic acid is derived from almonds and is a larger molecule than other AHAs used in skin care (e.g., glycolic acid). Thus, it tends to penetrate more slowly into the skin, making it less irritating to the skin than other AHAs. At low concentrations (e.g., 6 wt. % or less), it does not cause visible peeling of the skin, is safe for home use, and is well tolerated by patients of all skin types. Accordingly, mandelic acid is a viable option for topical application to improve skin quality. See generally, e.g., S. W. Jacobs, et al., “Effects of Topical Mandelic Acid Treatment on Facial Skin Viscoelasticity,” 34 Facial Plast. Surg. 651-56 (2018).

The one or more AHAs (e.g., mandelic acid) is present in the topical composition at any suitable concentration suitable for achieving a therapeutic benefit (e.g., decreasing cellular senescence). In some embodiments, the one or more AHAs (e.g., mandelic acid) is present in the topical composition at a concentration, relative to the total weight of the topical composition, of at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.15 wt. %, at least about 0.20 wt. %, at least about 0.25 wt. %, at least about 0.30 wt. %, at least about 0.35 wt. %, at least about 0.40 wt. %, at least about 0.45 wt. %, and less than 0.50 wt. %, or any range or value therein between.

In some embodiments, the one or more AHAs (e.g., mandelic acid) is present in the topical composition at a concentration, relative to the total weight of the topical composition, of less than 0.50 wt. %, less than or equal to about 0.45 wt. %, less than or equal to about 0.40 wt. %, less than or equal to about 0.35 wt. %, less than or equal to about 0.30 wt. %, less than or equal to about 0.25 wt. %, less than or equal to about 0.20 wt. %, less than or equal to about 0.15 wt. %, less than or equal to about 0.10 wt. %, less than or equal to about 0.09 wt. %, less than or equal to about 0.08 wt. %, less than or equal to about 0.07 wt. %, less than or equal to about 0.06 wt. %, less than or equal to about 0.05 wt. %, or any range or value therein between.

In some embodiments, the one or more AHAs (e.g., mandelic acid) is present in the topical composition at a concentration, relative to the total weight of the topical composition, of at least about 0.01 wt. % and less than 0.5 wt. %, about 0.01 wt. % to about 0.4 wt. %, about 0.01 wt. % to about 0.3 wt. %, about 0.01 wt. % to about 0.2 wt. %, about 0.01 wt. % to about 0.1 wt. %, about 0.01 wt. % to about 0.05 wt. %, about 0.01 wt. % to about 0.04 wt. %, about 0.01 wt. % to about 0.03 wt. %, 0.05 wt. % to about 0.5 wt. %, about 0.05 wt. % to about 0.4 wt. %, about 0.05 wt. % to about 0.3 wt. %, about 0.05 wt. % to about 0.2 wt. %, about 0.05 wt. % to about 0.1 wt. %, or any range or value therein.

In some embodiments, the one or more AHAs (e.g., mandelic acid) is present in the topical composition at concentrations lower than recommended for formulating the one or more AHAs into topical compositions (e.g., recommended concentration of about 0.5 wt. % to 6 wt. % for mandelic acid, to brighten skin). The present inventors surprisingly discovered that low concentrations of AHAs, and mandelic acid in particular, e.g., less than 0.5 wt. %, afford numerous therapeutic benefits, discussed below (see Methods of Use). At the same time, low concentrations of AHAs achieve desirable acidic pH values for topical compositions (e.g., 4 to 6, 3 to 5, or 4 to 5) while reducing or eliminating undesirable effects such as inflammation and skin irritation associated with use at higher doses.

The present inventors also surprisingly discovered that low doses of AHAs (e.g., mandelic acid) achieve synergistic activity with low doses of Centella asiatica extract, such as synergistic protection from products of senescent cells and decreased inflammation in the epidermis with increased cell cycle activity in the dermis. This activity implies synergy in reducing cellular senescence, which was unexpected and unknown in the art at the time.

The Centella asiatica extract and the AHA are present in any weight ratio suitable to achieve the above-mentioned synergistic activity. In some embodiments, the Centella asiatica extract and the AHA are present in a weight ratio of about 1:2 to 1:20, about 1:5 to 1:15, about 1:6 to 1:14, about 1:7 to about 1:13, about 1:8 to about 1:12, or any range or value therein. In some embodiments, the Centella asiatica extract and the AHA are present in a weight ratio of about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, or less.

In some embodiments, the triterpenes and the AHA are present in a weight ratio of about 1:2 to 1:400, about 1:5 to 1:200, about 1:6 to 1:100, about 1:7 to about 1:50, about 1:8 to about 1:20, about 1:10 to 1:15, or any range or value therein. In some embodiments, the triterpenes and the AHA are present in a weight ratio of about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:25, about 1:30, about 1:35, about 1:40, about 1:45, about 1:50, about 1:60, about 1:70, about 1:80, about 1:90, about 1:100, about 1:150, about 1:200, about 1:300, about 1:400 or less.

### Antioxidants

UV irradiation induces oxidative stress, which is detrimental to cellular functions and can negatively affect cell survival. Antioxidants can alleviate these oxidative processes by acting against the formation or propagation of reactive oxygen species, which will improve cellular function and survival.

In some embodiments, topical compositions according to the present disclosure comprise one or more antioxidants. In some embodiments, the one or more antioxidants comprise(s) tocopherol, tocopheryl acetate, hydroxyacetophenone, butylated hydroxyanisole (BHA), butylated hydroxytoluene (BHT), t-butyl hydroquinone (TBHQ), propyl gallate, tocotrienols, ascorbyl palmitate, Rosmarinus officinalis (rosemary) leaf extract, or a combination thereof. In some embodiments, the one or more antioxidants comprise(s) tocopherol, tocopheryl acetate, or a combination thereof. In some embodiments, the one or more antioxidants comprise(s) tocopherol. In some embodiments, the one or more antioxidants comprise(s) tocopheryl acetate. In some embodiments the one or more antioxidants is present separately from (e.g., in addition to) any antioxidants which may be present in the liposomes or liposomes present in the composition. In some embodiments, the one or more antioxidants is present in an oily phase that is prepared separately from the liposomes or liposomes present in the composition.

In some embodiments, the one or more antioxidants may be present, individually or collectively, at a concentration by weight, relative to the total weight of the composition, of at least about 0.001 wt. %, at least about 0.002 wt. %, at least about 0.003 wt. %, at least about 0.004 wt. %, at least about 0.005 wt. %, at least about 0.006 wt. %, at least about 0.007 wt. %, at least about 0.008 wt. %, at least about 0.009 wt. %, at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, or any range or value therein between. In some embodiments the one or more antioxidants is present at these concentrations separately from (e.g., in addition to) any antioxidants which may be present in the liposomes or liposomes present in the composition.

In some embodiments, the one or more antioxidants may be present, individually or collectively, at a concentration by weight, relative to the total weight of the composition, of no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, no greater than about 2.5 wt. %, no greater than about 2.0 wt. %, no greater than about 1.5 wt. %, no greater than about 1.0 wt. %, no greater than about 0.9 wt. %, no greater than about 0.8 wt. %, no greater than about 0.7 wt. %, no greater than about 0.6 wt. %, no greater than about 0.5 wt. %, no greater than about 0.4 wt. %, no greater than about 0.3 wt. %, no greater than about 0.2 wt. %, no greater than about 0.1 wt. %, no greater than about 0.09 wt. %, no greater than about 0.08 wt. %, no greater than about 0.07 wt. %, no greater than about 0.06 wt. %, no greater than about 0.05 wt. %, no greater than about 0.04 wt. %, no greater than about 0.03 wt. %, no greater than about 0.02 wt. %, no greater than about 0.01 wt. %, or any range or value therein between. In some embodiments the one or more antioxidants is present at these concentrations separately from (e.g., in addition to) any antioxidants which may be present in the liposomes or liposomes present in the composition.

In some embodiments, the one or more antioxidants may be present, individually or collectively, at a concentration by weight, relative to the total weight of the composition, of about 0.001 wt. %, about 0.002 wt. %, about 0.005 wt. %, about 0.008 wt. %, about 0.01 wt. %, about 0.02 wt. %, about 0.05 wt. %, about 0.08 wt. %, about 0.1 wt. %, about 0.2 wt. %, about 0.5 wt. %, about 0.8 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, or any range or value therein between. In some embodiments the one or more antioxidants is present at these concentrations separately from (e.g., in addition to) any antioxidants which may be present in the delivery vehicles (e.g., liposomes) present in the composition.

In some embodiments, the one or more antioxidants is present in the composition, individually or collectively, at a concentration by weight, relative to the total weight of the composition, of about 0.001 wt. % to about 10 wt. %, about 0.01 wt. % to about 10 wt. %, about 0.1 wt. % to about 10 wt. %, about 1 wt. % to about 10 wt. %, about 0.001 wt. % to about 1 wt. %, about 0.01 wt. % to about 1 wt. %, about 0.1 wt. % to about 1 wt. %, about 0.001 wt. % to about 0.1 wt. %, about 0.01 wt. % to about 0.1 wt. %, about 0.01 wt. % to about 10 wt. %, about 0.01 wt. % to about 1 wt. %, about 0.01 wt. % to about 0.1 wt. %, about 0.1 wt. % to about 1 wt. %, or any range or value therein between. In some embodiments the one or more antioxidants is present at these concentrations separately from (e.g., in addition to) any antioxidants which may be present in the delivery vehicles (e.g., liposomes) present in the composition.

### Humectants/Emollients

In some embodiments, a topical composition according to the present disclosure comprises one or more humectants and/or emollients. By way of non-limiting example, in some embodiments, the one or more humectants and/or emollients may comprise polyols, such as polyols having from 2 to 20 carbon atoms, including glycerol (glycerin); glycol derivatives (e.g., propylene glycol, butylene glycol, pentylene glycol, hexylene glycol, dipropylene glycol, diethylene glycol, caprylyl glycol); and mixtures thereof. In some embodiments, the humectants and/or emollients may comprise glycerin sorbitol; sugars (e.g., glucose, lactose, etc.); alkoxylated glucose derivatives; glucose ethers; panthenols (e.g., D-panthenol, DL-panthenol); polyethylene glycols (PEGs); urea; sodium hyaluronate; caprylyl glycol; pantolactone; caprylic/capric triglyceride; coco-caprylate/caprate; pantolactone; squalane; soluble chitosan; hexanetriol; amino acids (e.g., serine, citrulline, arginine, asparagine or alanine); alpha-hydroxy acids; salicylic acid; hyaluronic acid (HA); sodium lactate; sodium pyroglutamic acid (sodium PCA); aloe vera gel; or combinations thereof.

In some embodiments, the one or more humectants and/or emollients may be present at a concentration, individually or collectively, relative to the total weight of the composition, of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, or any range or value therein between.

In some embodiments, the one or more humectants and/or emollients may be present at a concentration, individually or collectively, relative to the total weight of the composition, of no greater than about 30 wt. %, no greater than about 25 wt. %, no greater than about 20 wt. %, no greater than about 15 wt. %, no greater than about 14 wt. %, no greater than about 13 wt. %, no greater than about 12 wt. %, no greater than about 11 wt. %, no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, or any range or value therein between.

In some embodiments, the one or more humectants and/or emollients may be present at a concentration, individually or collectively, relative to the total weight of the composition, of about 0.1 wt. %, about 0.2 wt. %, about 0.5 wt. %, about 0.8 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 15 wt. %, about 20 wt. %, about 25 wt. %, about 30 wt. %, or any range or value therein between.

In some embodiments, the one or more humectants and/or emollients may be present at a concentration, individually or collectively, relative to the total weight of the composition, of about 0.1 wt. % to about 30 wt. %, about 0.1 wt. % to about 25 wt. %, about 0.1 wt. % to about 20 wt. %, about 0.1 wt. % to about 15 wt. %, about 0.1 wt. % to about 14 wt. %, about 0.1 wt. % to about 13 wt. %, about 0.1 wt. % to about 12 wt. %, about 0.1 wt. % to about 11 wt. %, about 0.1 wt. % to about 10.0 wt. %, about 0.1 wt. % to about 8 wt. %, about 0.1 wt. % to about 5 wt. %, about 0.1 to about 4 wt. %, about 0.1 wt. % to about 3 wt. %, about 0.1 wt. % to about 2 wt. %, about 0.1 wt. % to about 1 wt. %, about 0.5 wt. % to about 30 wt. %, about 0.5 wt. % to about 25 wt. %, about 0.5 wt. % to about 20 wt. %, about 0.5 wt. % to about 15 wt. %, about 0.5 wt. % to about 10.0 wt. %, about 0.5 wt. % to about 8 wt. %, about 0.5 wt. % to about 5 wt. %, about 0.5 to about 4 wt. %, about 0.5 wt. % to about 3 wt. %, about 0.5 wt. % to about 2 wt. %, about 0.5 to about 1 wt. %, about 1 wt. % to about 30 wt. %, about 1 wt. % to about 25 wt. %, about 1 wt. % to about 20 wt. %, about 1 wt. % to about 15 wt. %, about 1 wt. % to about 10 wt. %, about 1 wt. % to about 8 wt. %, about 1 wt. % to about 5 wt. %, about 1 wt. % to about 4 wt. %, about 1 wt. % to about 3 wt. %, about 1 wt. % to about 2 wt. %, about 3 wt. % to about 30 wt. %, about 3 wt. % to about 25 wt. %, about 3 wt. % to about 20 wt. %, about 3 wt. % to about 15 wt. %, about 1 wt. % to about 10 wt. %, about 3 wt. % to about 8 wt. %, about 3 wt. % to about 5 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %, about 5 wt. % to about 8 wt. %, or any range or value therein.

### Emulsifiers

Topical compositions according to the present disclosure may comprise one or more emulsifiers to stabilize the aqueous phase and/or oily phase of the emulsion. Suitable emulsifiers for use in the topical compositions include anionic, cationic, nonionic, and zwitterionic surfactants.

In some embodiments, the emulsifiers comprise polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, polyoxyethylene sorbitan fatty acid esters, polyoxyethylene ethers of higher saturated fatty alcohols, and alkylpolyglucoside emulsifiers. In some embodiments, the emulsifiers comprise acrylic acid polymers (e.g., Acrylates/C10-30 Alkyl Acrylate Crosspolymer, such as that sold under the name PEMULEN™ EZ-4U). In some embodiments, the emulsifiers comprise potassium cetyl phosphate. In some embodiments, the emulsifiers comprise methyl glucose sesquistearate.

In some embodiments, the emulsifiers may be present at a concentration, individually or collectively, relative to the total weight of the composition, of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.2 wt. %, at least about 2.5 wt. %, at least about 2.8 wt. %, at least about 3.0 wt. %, at least about 3.2 wt. %, at least about 3.5 wt. %, at least about 3.8 wt. %, at least about 4.0 wt. %, at least about 4.2 wt. %, at least about 4.5 wt. %, at least about 4.8 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10.0 wt. %, or any range or value therein between.

In some embodiments, the emulsifiers may be present at a concentration, individually or collectively, relative to the total weight of the composition, of no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, or any range or value therein between.

In some embodiments, the emulsifiers may be present at a concentration, individually or collectively, relative to the total weight of the composition, of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, or any range or value therein.

In some embodiments, the emulsifiers may be present at a concentration, individually or collectively, relative to the total weight of the composition, of about 0.1 wt. % to about 10 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, 0.1 wt. % to about 0.5 wt. %, about 0.5 wt. % to about 10 wt. %, about 0.5 wt. % to about 5 wt. %, about 0.5 wt. % to about 4 wt. %, about 0.5 wt. % to about 3 wt. %, about 0.5 wt. % to about 2 wt. %, about 0.5 wt. % to about 1 wt. %, about 1 wt. % to about 10 wt. %, about 1 wt. % to about 5 wt. %, about 1 wt. % to about 4 wt. %, about 1 wt. % to about 3 wt. %, about 1 wt. % to about 2 wt. %, about 2 wt. % to about 10 wt. %, about 2 wt. % to about 5 wt. %, about 3 wt. % to about 10 wt. %, about 3 wt. % to about 5 wt. %, about 5 wt. % to about 10 wt. %, or any range or value therein.

### Oils

In some embodiments, a topical composition according to the present disclosure comprises one or more oils. In some embodiments (e.g., when the composition is in the form of an emulsion such as an oil-in-water or water-in-oil emulsion), the composition comprises an oily phase comprising one or more oils. By way of non-limiting example, the one or more oils may comprise vegetable oils, mineral oils, animal oils, or synthetic waxes, oils or butters, and mixtures thereof. In some embodiments, the oils may comprise: one or more mineral oils (e.g., PRIMOL 352®, MARCOL 82®, and MARCOL 152® sold by Esso); one or more vegetable oils (e.g., almond oil, sweet almond oil, palm oil, soybean oil, sesame oil, sunflower oil, olive oil, etc.); hydrogenated vegetable oils; one or more animal oils or substitutes of vegetable origin (e.g., lanolin, squalene or fish oil and derivatives thereof, such as perhydrosqualene, e.g., sold as SOPHIDERM® by Sophim); one or more synthetic oils (e.g., cetearyl isononanoate, such as CETIOL SN PH® by Cognis France, isononyl isononanoate, such as DUB ININ® sold by Stéarinerie Dubois, diisopropyl adipate, such as CRODAMOL DA® by Croda, isopropyl palmitate, such as CRODAMOL IPP® by Croda, caprylic/capric triglyceride, such as MIGLYOL 812® sold by Univar, hydrogenated polyisobutene, such as PARLEAM® products by Rossow); one or more silicone oils (e.g., dimethicone, such as Q7-9120 Silicone Fluid®, with a viscosity of 20 cSt to 12 500 cSt, by Dow Corning, cyclomethicone, such as ST-Cyclomethicone 5NF®, by Dow Corning, or DC 9045 Elastomer Blend®, by Dow Corning); or any combination thereof.

In some embodiments, the one or more oils be present at a concentration, individually or collectively, relative to the total weight of the composition, of at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, at least about 15.0 wt. %, at least about 20.0 wt. %, or any range or value therein between.

In some embodiments, the one or more oils may be present at a concentration, individually or collectively, relative to the total weight of the composition, of no greater than about 35.0 wt. %, no greater than about 30 wt. %, no greater than about 25 wt. %, no greater than about 20 wt. %, no greater than about 19 wt. %, no greater than about 18 wt. %, no greater than about 17 wt. %, no greater than about 16 wt. %, no greater than about 15 wt. %, no greater than about 14 wt. %, no greater than about 13 wt. %, no greater than about 12 wt. %, no greater than about 11 wt. %, no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, or any range or value therein between.

In some embodiments, the one or more oils may be present at a concentration, individually or collectively, relative to the total weight of the composition, of about 0.5 wt. %, about 0.8 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 35 wt. %, or any range or value therein between.

In some embodiments, the one or more oils may be present at a concentration, individually or collectively, relative to the total weight of the composition, of about 0.5 wt. % to about 35 wt. %, about 0.5 wt. % to about 30 wt. %, about 0.5 wt. % to about 25 wt. %, about 0.5 wt. % to about 20 wt. %, about 0.5 wt. % to about 15 wt. %, about 0.5 wt. % to about 10.0 wt. %, about 0.5 wt. % to about 5 wt. %, about 0.5 wt. % to about 4 wt. %, about 0.5 wt. % to about 3 wt. %, about 0.5 wt. % to about 2 wt. %, about 0.5 to about 1 wt. %, about 1 wt. % to about 30 wt. %, about 1 wt. % to about 25 wt. %, about 1 wt. % to about 20 wt. %, about 1 wt. % to about 15 wt. %, about 1 wt. % to about 10 wt. %, about 1 wt. % to about 8 wt. %, about 1 wt. % to about 5 wt. %, about 1 wt. % to about 4 wt. %, about 1 wt. % to about 3 wt. %, about 1 wt. % to about 2 wt. %, about 3 wt. % to about 30 wt. %, about 3 wt. % to about 20 wt. %, about 3 wt. % to about 15 wt. %, about 3 wt. % to about 10 wt. %, about 3 wt. % to about 8 wt. %, about 3 wt. % to about 5 wt. %, about 5 wt. % to about 10 wt. %, about 5 wt. % to about 8 wt. %, or any range or value therein.

### Additional Fatty Phase Components

Compositions according to the present disclosure may comprise an oily or fatty phase. In some embodiments, the oily or fatty phase comprises oils and additional components (e.g., waxes, fatty alcohols, fatty acids, esters of carboxylic acids or diacids, alkyl esters of fatty acids or diacids, alkenyl esters of fatty acids or diacids, mineral oils, petrolatum, etc.).

In some embodiments, topical compositions according to the present disclosure comprise one or more waxes. In some embodiments, the waxes are selected from lanolin and derivatives thereof including lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, amide waxes, microcrystalline waxes, hydrogenated vegetable oils, Oryz sativa (rice) bran wax, Helianthus annuus (sunflower) seed wax, paraffins, ceresin, and combinations thereof. Also usable as waxes are wax esters such as beeswax (e.g., synthetic beeswax), spermaceti, myristyl myristate and stearyl stearate; beeswax derivatives, e.g., polyoxyethylene sorbitol beeswax; and vegetable waxes including carnauba and candelilla waxes.

In some embodiments, topical compositions according to the present disclosure comprise one or more fatty acids (e.g., pelargonic, lauric, myristic, palmitic, stearic, isostearic, hydroxystearic, oleic, linoleic, ricinoleic, arachidic, behenic, erucic acids, and combinations thereof). In some embodiments, topical compositions according to the present disclosure comprise one or more fatty alcohols (e.g., lauryl, myristyl, cetyl, hexadecyl, stearyl, isostearyl, hydroxystearyl, oleyl, ricinoleyl, behenyl, and erucyl alcohols, and combinations thereof, such as cetostearyl alcohol (i.e., cetearyl alcohol)), as well as 2-octyl dodecanol.

In some embodiments, topical compositions according to the present disclosure comprise esters of carboxylic acids or diacids (e.g., methyl, isopropyl, and butyl esters of fatty acids). In some embodiments, topical compositions according to the present disclosure comprise alkyl esters (e.g., hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate). In some embodiments, topical compositions according to the present disclosure comprise alkenyl esters of fatty acids such as oleyl myristate, oleyl stearate, and oleyl oleate. In some embodiments, topical compositions according to the present disclosure comprise alkyl esters of diacids (e.g., diisopropyl adipate, diisohexyl adipate, bis(hexyldecyl) adipate, and diisopropyl sebacate).

In some embodiments, the additional fatty phase components (e.g., waxes, fatty alcohols, fatty acids, esters of carboxylic acids or diacids, alkyl esters of fatty acids or diacids, alkenyl esters of fatty acids or diacids) may be present at a concentration, individually or collectively, relative to the total weight of the composition, of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.2 wt. %, at least about 2.5 wt. %, at least about 2.8 wt. %, at least about 3.0 wt. %, at least about 3.2 wt. %, at least about 3.5 wt. %, at least about 3.8 wt. %, at least about 4.0 wt. %, at least about 4.2 wt. %, at least about 4.5 wt. %, at least about 4.8 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10.0 wt. %, or any range or value therein between.

In some embodiments, the additional fatty phase components (e.g., waxes, fatty alcohols, fatty acids, esters of carboxylic acids or diacids, alkyl esters of fatty acids or diacids, alkenyl esters of fatty acids or diacids) may be present at a concentration, individually or collectively, relative to the total weight of the composition, of no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, or any range or value therein between.

In some embodiments, the additional fatty phase components (e.g., waxes, fatty alcohols, fatty acids, esters of carboxylic acids or diacids, alkyl esters of fatty acids or diacids, alkenyl esters of fatty acids or diacids) may be present at a concentration, individually or collectively, relative to the total weight of the composition, of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, or any range or value therein.

In some embodiments, the additional fatty phase components (e.g., waxes, fatty alcohols, fatty acids, esters of carboxylic acids or diacids, alkyl esters of fatty acids or diacids, alkenyl esters of fatty acids or diacids) may be present at a concentration, individually or collectively, relative to the total weight of the composition, of about 0.1 wt. % to about 10 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, 0.1 wt. % to about 0.5 wt. %, about 0.5 wt. % to about 10 wt. %, about 0.5 wt. % to about 5 wt. %, about 0.5 wt. % to about 4 wt. %, about 0.5 wt. % to about 3 wt. %, about 0.5 wt. % to about 2 wt. %, about 0.5 wt. % to about 1 wt. %, about 1 wt. % to about 10 wt. %, about 1 wt. % to about 5 wt. %, about 1 wt. % to about 4 wt. %, about 1 wt. % to about 3 wt. %, about 1 wt. % to about 2 wt. %, about 2 wt. % to about 10 wt. %, about 2 wt. % to about 5 wt. %, about 3 wt. % to about 10 wt. %, about 3 wt. % to about 5 wt. %, about 5 wt. % to about 10 wt. %, or any range or value therein.

### Gelling Agents

In some embodiments, a topical composition according to the present disclosure comprises one or more gelling agents (also known as suspending agents or thickening agents). By way of non-limiting example, the one or more gelling agents may comprise ready-for-use mixtures (e.g., Polyacrylate-13/Polyisobutene/Polysorbate 20 sold by Seppic under the name SEPIPLUS 400®, or the Ammonium Acrylate/Acrylamide Copolymer/Polyisobutene/Polysorbate 20 mixture sold by Seppic under the name SEPIPLUS 265®, or Acrylamide/Sodium Acryloyldimethyl Taurate Copolymer/Isohexadecane/Polysorbate 80 sold by Seppic under the name SEVIULGEL™ 600); acrylic acid polymers (e.g., Acrylates/C10-30 Alkyl Acrylate Crosspolymer, such as that sold under the name PEMULEN™ EZ-4U); carbomers (e.g., ULTREZ 20®, ULTREZ 10®, CARBOPOL 1382®, CARBOPOL ETD2020NF® or AQUA SF1® sold by Lubrizol); polysaccharides (e.g., xanthan gum (such as XANTURAL 180® sold by Kelco), gellan gum (e.g., KELCOGEL® by Kelco), sclerotium gum (e.g., AMIGEL® by Alban Muller Industrie), guar gum and its derivatives (such as hydroxypropyl guar gum sold under the name JAGUAR HP-105® by Rhodia), cationic guar gums, pullulan, cellulose and cellulose derivatives (such as microcrystalline cellulose and sodium carboxymethyl cellulose, e.g., sold under the name BLANOSE CMC 7H4XF® by Hercules, hydroxypropylmethyl cellulose, e.g., sold under the name of METHOCEL E4M® Premium by Dow Chemical, hydroxyethyl cellulose, e.g., sold under the name of NATROSOL HHX 250® by Aqualon, methyl cellulose, carboxymethyl cellulose); magnesium aluminum silicates (e.g., VEEGUM K®, VEEGUM Plus®, or VEEGUM Ultra® sold by Vanderbilt); bentonite; modified starches (e.g., modified potato starch sold under the name of STRUCTURE SOLANACE®); carrageenans (e.g., the κ, λ, β and ω families, such as the VISCARIN® and GELCARIN® products sold by IMCD); polyvinyl alcohols (PVAs) (e.g., Polyvinyl Alcohol 40-88® sold by Merck); polyvinylpyrrolidones; carboxyvinyl polymers; acrylic acid/ethyl acrylate copolymers (e.g., CARBOPOLS®); polyacrylic acid polymers; polymethyacrylic acid polymers; polyvinyl acetate polymers, polyvinylchloride polymers; polyvinylidene chloride polymers; mixtures of polyethylene glycol and polyethylene glycol stearate or distearate; oleogels (e.g., trihydroxystearin or aluminum magnesium hydroxy stearate); non-ionic polymers; and any combination of the above.

In some embodiments, the one or more gelling agents may be present at a concentration, individually or collectively, relative to the total weight of the composition, of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.2 wt. %, at least about 1.5 wt. %, at least about 1.8 wt. %, at least about 2.0 wt. %, at least about 2.2 wt. %, at least about 2.5 wt. %, at least about 2.8 wt. %, at least about 3.0 wt. %, at least about 3.2 wt. %, at least about 3.5 wt. %, at least about 3.8 wt. %, at least about 4.0 wt. %, at least about 4.2 wt. %, at least about 4.5 wt. %, at least about 4.8 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10.0 wt. %, or any range or value therein between.

In some embodiments, the one or more gelling agents may be present at a concentration, individually or collectively, relative to the total weight of the composition, of no greater than about 10.0 wt. %, no greater than about 9.5 wt. %, no greater than about 9.0 wt. %, no greater than about 8.5 wt. %, no greater than about 8.0 wt. %, no greater than about 7.5 wt. %, no greater than about 7.0 wt. %, no greater than about 6.5 wt. %, no greater than about 6.0 wt. %, no greater than about 5.5 wt. %, no greater than about 5.0 wt. %, no greater than about 4.5 wt. %, no greater than about 4.0 wt. %, no greater than about 3.5 wt. %, no greater than about 3.0 wt. %, or any range or value therein between.

In some embodiments, the one or more gelling agents may be present at a concentration, individually or collectively, relative to the total weight of the composition, of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.2 wt. %, about 1.5 wt. %, about 1.8 wt. %, about 2.0 wt. %, about 2.2 wt. %, about 2.5 wt. %, about 2.8 wt. %, about 3.0 wt. %, about 3.2 wt. %, about 3.5 wt. %, about 3.8 wt. %, about 4.0 wt. %, about 4.2 wt. %, about 4.5 wt. %, about 4.8 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, or any range or value therein.

In some embodiments, the one or more gelling agents may be present at a concentration, individually or collectively, relative to the total weight of the composition, of about 0.1 wt. % to about 10 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, 0.1 wt. % to about 0.5 wt. %, about 0.5 wt. % to about 10 wt. %, about 0.5 wt. % to about 5 wt. %, about 0.5 wt. % to about 4 wt. %, about 0.5 wt. % to about 3 wt. %, about 0.5 wt. % to about 2 wt. %, about 0.5 wt. % to about 1 wt. %, about 1 wt. % to about 10 wt. %, about 1 wt. % to about 5 wt. %, about 1 wt. % to about 4 wt. %, about 1 wt. % to about 3 wt. %, about 1 wt. % to about 2 wt. %, about 2 wt. % to about 10 wt. %, about 2 wt. % to about 5 wt. %, about 3 wt. % to about 10 wt. %, about 3 wt. % to about 5 wt. %, about 5 wt. % to about 10 wt. %, or any range or value therein.

### Composition pH

A topical composition according to the present disclosure may have any suitable pH for ensuring chemical and physical stability of the composition and any active ingredients contained therein, non-irritation to the skin, and prevention of dry skin.

In some embodiments, an anti-inflammatory topical composition according to the present disclosure has a pH of between about 3.0 and about 9.0, between about 3.0 and about 8.5, between about 3.0 and about 8.0, between about 3.0 and about 7.5, between about 3.0 and about 7.0, between about 3.0 and about 6.5, between about 3.0 and about 6.0, between about 3.0 and about 5.5, between about 3.0 and about 5.0, between about 3.0 and about 4.5, between about 3.5 and about 9.0, between about 3.5 and about 8.5, between about 3.5 and about 8.0, between about 3.5 and about 7.5, between about 3.5 and about 7.0, between about 3.5 and about 6.5, between about 3.5 and about 6.0, between about 3.5 and about 5.5, between about 3.5 and about 5.0, between about 3.5 and about 4.5, between about 4.0 and about 9.0, between about 4.0 and about 8.5, between about 4.0 and about 8.0, between about 4.0 and about 7.5, between about 4.0 and about 7.0, between about 4.0 and about 6.5, between about 4.0 and about 6.0, between about 4.0 and about 5.5, between about 4.0 and about 5.0, between about 4.5 and about 9.0, between about 4.5 and about 8.5, between about 4.5 and about 8.0, between about 4.5 and about 7.5, between about 4.5 and about 7.0, between about 4.5 and about 6.5, between about 4.5 and about 6.0, between about 4.5 and about 6.0, between about 4.5 and about 5.5, between about 4.5 and about 5.0, or any range or value therein.

In some embodiments, an anti-inflammatory topical composition according to the present application may have a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1 about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, or any range or value therein between.

In some embodiments, the topical composition has an acidic pH, such as less than about 7, less than about 6.5, less than about 6.0, less than about 5.5, less than about 5.0, less than about 4.5, or any range or value therein between.

In particular, acidic topical compositions may be advantageous for treating elderly patients having dermatoporosis or atopic dermatitis. Skin treated with acidic emulsions (e.g., pH 4) have shown resistance to oxidative damage, increased barrier integrity, and reduced roughness and scaliness of the skin surface after several weeks of treatment. See, e.g., M. Luki\tilde{c}, et al., “Towards Optimal pH of the Skin and Topical Formulations: From the Current State of the Art to Tailored Products,” 8 Cosmetics 69 (2021); A. Kilic, et al. “Skin Acidification With a Water-in-Oil Emulsion (pH 4) Restores Disrupted Epidermal Barrier and Improves Structure of Lipid Lamellae in the Elderly,” 46 J. Dermatol. 457-65 (2019).

### pH Adjusting Agents

In some embodiments, a topical composition may include one or more pH adjusting agents suitable for adjusting the pH of the composition to be in any of the ranges discussed above. In some embodiments, the pH adjusting agent may comprise one or more suitable mineral acids (e.g., hydrochloric acid, nitric acid, phosphoric acid, phosphorous acid, sulfuric acid, etc.), carboxylic acids (e.g., citric acid, glycolic acid, lactic acid, maleic acid, malic acid, succinic acid, glutaric acid, benzoic acid, malonic acid, salicylic acid, gluconic acid, etc.), polymeric acids (e.g., straight-chain poly(acrylic) acid and its copolymers, such as maleic-acrylic, sulfonic-acrylic, and styrene-acrylic copolymers), cross-linked polyacrylic acids, poly(methacrylic) acids, carageenic acid, alginic acid, etc.), and any combination thereof.

The pH may be raised or made more alkaline by addition of any suitable alkaline pH adjusting agent (e.g., sodium hydroxide, potassium hydroxide, sodium carbonate, sodium bicarbonate, etc.). In some embodiments, the one or more pH adjusting agents may comprise: ammonia; mono-, di-, and tri-alkyl amines (e.g., trimethylamine); mono-, di-, and tri-alkanolamines (e.g., monoethanolamine, diethanolamine, triethanolamine, isopropanolamine, diisopropanolamine, and triisopropanolamine); alkali metal and alkaline earth metal hydroxides (e.g., sodium hydroxide, potassium hydroxide, lithium hydroxide, etc.); alkali metal and alkaline earth metal silicates; and other pH adjusters (e.g., aminomethylpropanol (AMP-95), tetrahydroxypropylethylenediamine, ETHOMEEN® C-25 (PEG-15 cocoamine), etc.).

In some embodiments, the one or more pH adjusting agents comprises a buffering agent. A buffering agent is a chemical compound that is or compounds that are added to a solution to allow that solution to resist changes in pH as a result of either dilution or small additions of acids or bases. Effective buffer systems employ solutions which contain large and approximately equal concentrations of a conjugate acid-base pair (or buffering agents). A buffering agent employed herein may be any such chemical compound(s) which is pharmaceutically acceptable, including but not limited to salts (conjugates acids and/or bases) of phosphates and citrates. In some aspects, the buffering agent comprises phosphate buffered saline (PBS) or an alternative phosphate buffer.

### Additional Ingredients

Topical compositions according to the present disclosure may comprise a broad range of optional ingredients or additives. The CTFA International Cosmetic Ingredient Dictionary, Fifteenth Edition, 2014, which is incorporated by reference herein in its entirety, describes a wide variety of non-limiting cosmetic and pharmaceutical ingredients commonly used in the skin care industry, which are suitable for use in the topical compositions of the present disclosure. Non-limiting examples of genera of such ingredients include: abrasives, anti-acne agents, anticaking agents (e.g., silica, distarch phosphate, etc.), binders, biological additives, bulking agents, chelating agents (e.g., disodium EDTA), chemical additives; colorants; cosmetic astringents, cosmetic biocides, denaturants, drug astringents, external analgesics, film formers, fragrance components, opacifying agents, plasticizers, preservatives (e.g., phenoxyethanol, ethylhexylglycerin, etc.), propellants, reducing agents, skin bleaching agents, skin-conditioning agents, skin protectants, solvents (e.g., ethanol, 1,2-hexanediol, etc.), foam boosters, hydrotropes, solubilizing agents, suspending agents (nonsurfactant), sunscreen agents, ultraviolet light absorbers, and viscosity increasing agents (aqueous and nonaqueous), solubilizing agents, sequestrants, and keratolytics, plant extracts, dipeptides, tripeptides (e.g., tripeptide-1) and derivatives thereof, tetrapeptides (e.g., tetrapeptide-2 and derivatives thereof), hexapeptides (e.g., hexapeptide-11, hexapeptide-12, hexapeptide-38, and derivatives thereof), octapeptides (e.g., octapeptide-45 and derivatives thereof), and any combination thereof.

When present, such additional ingredients are preferably present at relatively low concentrations, such as from about 0.001 wt. % to about 5 wt. %, about 0.001 wt. % to about 1 wt. %, about 0.001 wt. % to about 0.5 wt. %, about 0.001 wt. % to about 0.1 wt. %, about 0.001 wt. % to about 0.05 wt. %, about 0.001 wt. % to about 0.01 wt. %, or any range or value therein between.

### Water

A topical composition according to the present disclosure may comprise water at a concentration by weight, relative to the total weight of the composition, of about 0 wt. % to about 98 wt. %, about 5 wt. % to about 95 wt. %, about 10 wt. % to about 90 wt. %, about 15 wt. % to about 85 wt. %, about 20 wt. % to about 80 wt. %, about 25 wt. % to about 75 wt. %, about 30 wt. % to about 70 wt. %, about 35 wt. % to about 65 wt. %, or about 40 wt. % to about 60 wt. %, or any range or value therein. In some embodiments, the water is present at a concentration by weight, relative to the total weight of the composition, of about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 96 wt. %, about 97 wt. %, about 98 wt. %, about 99 wt. %, or any range or value therein between.

### Composition Forms

A topical composition according to the present disclosure may be in any galenical form that ensures the composition is stable, non-irritating to the skin, non-drying to the skin, and/or pleasant and easy to apply. In some embodiments, the topical composition is an emulsion (e.g., oil-in water emulsion or water-in-oil emulsion), a gel, a cream, a cream-gel, a solution, suspension, lotion, milk, ointment, salve, foam (e.g., aerosol or self-foaming composition), balm, paste, or sachet. In some embodiments, the topical composition is an emulsion. In some embodiments, the topical composition is a gel. In some embodiments, the topical composition is a cream. In some embodiments, the topical composition is a galenical form suitable for a pump dispenser. In some embodiments, the anti-inflammatory composition is formulated for leave-on (as opposed to “rinse-off”) application. In some embodiments, the topical composition is an oil-in-water (O/W) emulsion.

### Methods of Use

In an aspect, the present disclosure relates to a method of treating skin affected by dermatoporosis, the method comprising: administering a topical composition according to the present disclosure to the affected skin.

In an aspect, the present disclosure relates to a method of increasing fibroblast cell turnover in mammalian skin, the method comprising: administering a topical composition according to the present disclosure to the mammalian skin. In any embodiment described herein, the mammalian skin may comprise human skin.

In an aspect, the present disclosure relates to a method of reducing the number of senescent cells in mammalian dermis, the method comprising: administering a topical composition according to the present disclosure to the mammalian skin. In some embodiments, the method comprises reducing the number of senescent cells in the dermis and clearing senescent cells from the dermis to the epidermis.

In an aspect, the present disclosure relates to a method of reducing senescent cell activity in mammalian skin, the method comprising: administering a topical composition according to the present disclosure to the mammalian skin. In some embodiments, the method comprises reducing the expression and/or activity of p16 in mammalian skin.

In an aspect, the present disclosure relates to a method of reducing the senescence-associated secretory phenotype (SASP) in mammalian epidermal cells, the method comprising: administering a topical composition according to the present disclosure to the mammalian skin. In some aspects, the present disclosure relates to a method of modulating at least one SASP-associated gene in mammalian skin, the method comprising: administering a topical composition according to the present disclosure to the mammalian skin, wherein the at least one SASP-associated gene is selected from the group consisting of IL8, IL1B, HIST1H2BG, and UBE2C. In some embodiments, the modulating comprises downregulating one or more genes and/or upregulating one or more genes. In some embodiments, the modulating comprises downregulating one or more genes selected from the group consisting of IL8, IL1B, HIST1H2BG, and UBE2C. In some embodiments, the modulating comprises downregulating IL8. In some embodiments, the modulating comprises downregulating IL1B. In some embodiments, the modulating comprises downregulating HIST1H2BG. In some embodiments, the modulating comprises downregulating UBE2C. In some embodiments, the modulating is in keratinocytes.

In an aspect, the present disclosure relates to a method of modulating one or more inflammation-related genes in mammalian skin, the method comprising: administering a topical composition according to the present disclosure to the mammalian skin, wherein the at least one inflammation-related gene is selected from the group consisting of MMP2, HSPA8, CSF2RA, CXCL1, IL2RB, NFKBIA, and PTGS2. In some embodiments, the modulating comprises downregulating one or more genes and/or upregulating one or more genes. In some embodiments, the modulating comprises downregulating one or more genes selected from the group consisting of MMP2, HSPA8, CSF2RA, CXCL1, IL2RB, NFKBIA, and PTGS2. In some embodiments, the modulating comprises downregulating MMP2. In some embodiments, the modulating comprises downregulating HSPA8. In some embodiments, the modulating comprises downregulating CSF2RA. In some embodiments, the modulating comprises downregulating CXCL1. In some embodiments, the modulating comprises downregulating IL2RB. In some embodiments, the modulating comprises downregulating NFKBIA. In some embodiments, the modulating comprises downregulating PTGS2. In some embodiments, the gene is related to skin inflammation. In some embodiments, the modulating is in keratinocytes.

In an aspect, the present disclosure relates to a method of modulating expression of one or more genes related to p15 activity in mammalian skin, the method comprising: administering a topical composition according to the present disclosure to the mammalian skin, wherein the one or more genes related to p15 activity comprises CDKN2B. In some embodiments, the modulating is upregulating. In some embodiments, the method comprises upregulating the expression of CDKN2B in mammalian skin. In some embodiments, the modulating is in keratinocytes.

In an aspect, the present disclosure relates to a method of modulating the expression of at least one cell cycle gene in mammalian dermal fibroblasts, the method comprising: administering a topical composition according to the present disclosure to the mammalian skin, wherein the at least one cell cycle gene in mammalian fibroblasts is selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH. In some embodiments, the modulating comprises downregulating one or more genes and/or upregulating one or more genes. In some embodiments, the modulating comprises upregulating one or more genes selected from TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH. In some embodiments, the modulating comprises upregulating TK1. In some embodiments, the modulating comprises upregulating NDC80. In some embodiments, the modulating comprises upregulating HMMR. In some embodiments, the modulating comprises upregulating KIF2C. In some embodiments, the modulating comprises upregulating UBEC. In some embodiments, the modulating comprises upregulating LMNB1. In some embodiments, the modulating comprises upregulating CABLES1. In some embodiments, the modulating comprises upregulating MCM10. In some embodiments, the modulating comprises upregulating H2AFX. In some embodiments, the modulating comprises upregulating CASC5. In some embodiments, the modulating comprises upregulating ESCO2. In some embodiments, the modulating comprises upregulating ERCC6L. In some embodiments, the modulating comprises upregulating BUB1. In some embodiments, the modulating comprises upregulating BIRC5. In some embodiments, the modulating comprises upregulating CCNE2. In some embodiments, the modulating comprises upregulating GTSE1. In some embodiments, the modulating comprises upregulating CDCA8. In some embodiments, the modulating comprises upregulating WEE1. In some embodiments, the modulating comprises upregulating CDC45. In some embodiments, the modulating comprises upregulating CDK1. In some embodiments, the modulating comprises upregulating CCNB2. In some embodiments, the modulating comprises upregulating CLSPN. In some embodiments, the modulating comprises upregulating BUB1B. In some embodiments, the modulating comprises upregulating PKMYT1. In some embodiments, the modulating comprises upregulating KIF23. In some embodiments, the modulating comprises upregulating TPX2. In some embodiments, the modulating comprises upregulating KIF20A. In some embodiments, the modulating comprises upregulating NCAPH.

In an aspect, the present disclosure relates to a method of downregulating at least one transcription factor of the AP-1 complex, the method comprising: administering a topical composition of the present disclosure to mammalian skin. In some embodiments, the transcription factor of the AP-1 complex is at least one selected from JunB and FosL2.

In an aspect, which may be combined with any other aspect or embodiment, the present disclosure relates to a method of activating cell cycle progression, the method comprising: administering a topical composition of the present disclosure to mammalian skin, wherein the composition upregulates CCNE2, CCNB1, or a combination thereof.

In an aspect, the present disclosure relates to a method of increasing dermal fibroblast cell turnover in mammalian skin, the method comprising: administering a topical composition according to the present disclosure to the affected skin.

In an aspect, the present disclosure relates to a method of stimulating collagen production in mammalian skin, the method comprising: administering a topical composition according to the present disclosure to the affected skin.

In any aspect or embodiment, the modulating (upregulating or downregulating) of gene expression may be measured in terms of -fold change relative to baseline expression or expression in untreated cells. In some embodiments, upregulating is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 15-fold, at least 20-fold, or greater, or any range or value therein between.

In some embodiments, down-regulating is at least −0.1-fold, at least −0.2-fold, at least −0.3-fold, at least −0.4-fold, at least −0.5-fold, at least −0.6-fold, at least −0.7-fold, at least −0.8-fold, at least −0.9-fold, at least −1.0-fold, at least −1.1-fold, at least −1.2-fold, at least −1.3-fold, at least −1.4-fold, at least −1.5-fold, at least −1.6-fold, at least −1.7-fold, at least −1.8-fold, at least −1.9-fold, at least −2.0-fold, at least −2.1-fold, at least −2.2-fold, at least −2.3-fold, at least −2.4-fold, at least −2.5-fold, at least −2.6-fold, at least −2.7-fold, at least −2.8-fold, at least −2.9-fold, at least −3.0-fold, at least −3.1-fold, at least −3.2-fold, at least −3.3-fold, at least −3.4-fold, at least −3.5-fold, at least −3.6-fold, at least −3.7-fold, at least −3.8-fold, at least −3.9-fold, at least −4.0-fold, at least −4.1-fold, at least −4.2-fold, at least −4.3-fold, at least −4.4-fold, at least −4.5-fold, at least −4.6-fold, at least −4.7-fold, at least −4.8-fold, at least −4.9-fold, at least −5.0-fold, at least −5.5-fold, at least −6.0-fold, at least −6.5-fold, at least −7.0-fold, at least −7.5-fold, at least −8.0-fold, at least −8.5-fold, at least −9.0-fold, at least −9.5-fold, at least −10.0-fold, at least −15-fold, at least −20-fold, or greater, or any range or value therein between.

Topical compositions according to the present disclosure may be used with various treatment regimens. In some instances, the topical compositions described herein are administered once per day, twice per day, three times per day, or more. In some instances, the topical compositions described herein are administered twice per day. The topical compositions described herein, in some embodiments, are administered daily, every day, every alternate day, five days per week, once per week, every other week, two weeks per month, three weeks per month, once per month, twice per month, three times per month, or more. In some embodiments, the topical compositions described herein are administered twice daily (e.g., morning and evening).

In some embodiments, the topical compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, the topical compositions described herein are administered twice daily for at least or about 1 week, 2 weeks, 3 weeks, 1 month, 2 months, 3 months, 4 months, 5 months, 6 months, or more. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more.

### EXAMPLES

### Example 1. Preparation of Topical Compositions

An exemplary topical composition according to the present disclosure is shown in Table 1 below.

TABLE 1
Exemplary Topical Compositions

| | | | Concentration (wt. %) | | |
|---|---|---|---|---|---|
| Phase | Raw Material | | A | B | C |
| A | Water | | qs. 100 | qs. 100 | qs. 100 |
| A | Acrylates/C10-30 Alkyl Acrylate | | 0.1-0.5 | 0.2-1.5 | 0.1-5.0 |
| | Crosspolymer | | | | |
| A | Disodium EDTA | | 0.05-0.2 | 0.05-0.5 | 0.02-1.0 |
| A | Glycerin | | 5.0-15.0 | 3.0-20.0 | 1.0-20.0 |
| B | Sunflower Oil and/or Sweet Almond Oil | | 12.0-15.0 | 10.0-15.0 | 10.0-20.0 |
| B | Hydrogenated Vegetable Oil | | 1.5-2.5 | 1.0-3.0 | 0.5-5.0 |
| B | Cetearyl Alcohol | | 1.0-5.0 | 0.5-5.0 | 1.0-10.0 |
| B | Methyl Glucose Sequistearate | | 0.5-5.0 | 0.1-5.0 | 0.1-10.0 |
| B | Synthetic Beeswax | | 1.0-5.0 | 0.5-5.0 | 0.5-10.0 |
| B | Tocopheryl Acetate | | 0.1-1.0 | 0.1-2.0 | 0.05-5.0 |
| C | Potassium Cetyl Phosphate | | 0.1-1.0 | 0.1-2.0 | 0.05-5.0 |
| D | Dimethicone | | 1.0-5.0 | 0.5-5.0 | 0.1-8.0 |
| E | Sodium Hydroxide Solution in Water | | as needed | as needed | as needed |
| F | Phenoxyethanol | | 0.1-1.0 | 0.1-2.0 | 0.05-3.0 |
| F | Ethylhexylglycerin | | 0.01-0.5 | 0.01-1.0 | 0.01-3.0 |
| G | PRO-LIPO ™ NEO | Propanediol | 0.1-1.0 | 0.05-3.0 | 0.02-5.0 |
| | | Lecithin | | | |
| | | Tocopherol | | | |
| | | Sunflower Seed Oil | | | |

| | | | | |
|---|---|---|---|---|
| G | Water | 0.1-5.0 | 0.05-10.0 | 0.02-10.0 |
| G | Centella Asiatica Leaf Extract | 0.01-0.2 | 0.005-0.2 | 0.001-0.1 |
| | (HETEROSIDES, Seppic) | | | |
| H | Mandelic Acid | 0.01-0.5 | 0.005-0.5 | 0.001-0.5 |

Topical compositions according to Table 1 are prepared according to the following general procedure:

In a main tank, a gelling phase (Phase A) is prepared by adding gelling agent (e.g., acrylates/C10-30 alkyl acrylate crosspolymer), disodium EDTA, glycerin, and water, under stirring until completely dispersed. In a first annex tank, an oily phase (Phase B) is prepared by adding oils (e.g., sunflower seed oil, hydrogenated vegetable oil) and non-liquid fatty substances (e.g., cetearyl alcohol, synthetic beeswax) under stirring, followed by emulsifiers (e.g., methyl glucose sesquistearate), antioxidants (e.g., tocopheryl acetate), which are stirred until completely dispersed. Phase B is added to Phase A under stirring, followed by an emulsifying phase, Phase C (potassium cetyl phosphate), until an oil-in-water emulsion is observed. Next, the acrylate is neutralized by adding sodium hydroxide solution (e.g., to a pH of about 5-6). A preservative phase, Phase F (e.g., phenoxyethanol, ethylhexylglycerin), is then added under stirring until completely dispersed.

In a second annex tank, an active liposome phase (Phase G) is prepared by stirring a liposome mixture (e.g., PRO-LIPO™ NEO), water, and Centella asiatica leaf extract (e.g., HETEROSIDES) until the Centella asiatica leaf extract is completely dispersed in the liposome mixture. Phase G is then added to the pH-adjusted emulsion in the main tank under stirring until completely dispersed. Finally, AHA phase (Phase H) comprising mandelic acid is added to the main tank under stirring until completely dispersed and the pH is between 4 and 5, to obtain an oil-in-water emulsion comprising liposome-encapsulated Centella asiatica leaf extract and mandelic acid.

### Example 2. Cytotoxicity Study of Mandelic Acid and Centella asiatica Extract

To determine suitable dosing of Centella asiatica extract and mandelic acid, human primary adult dermal fibroblasts and human primary adult dermal keratinocytes were purchased from ZenBio (https://www.zen-bio.com/, Durham, NC) and plated as per the provided ZenBio protocols in the recommended media from ZenBio.

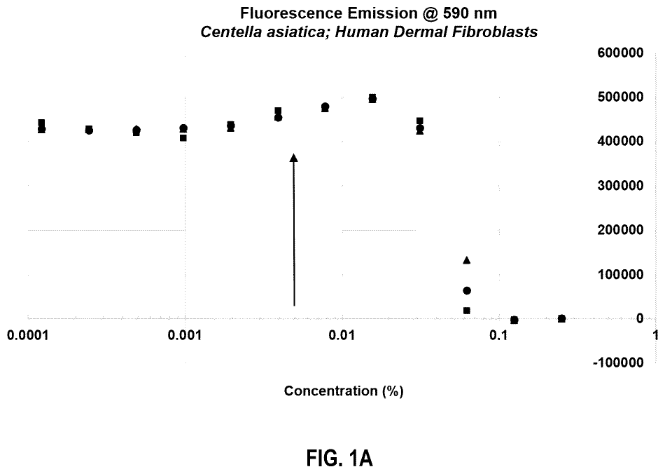

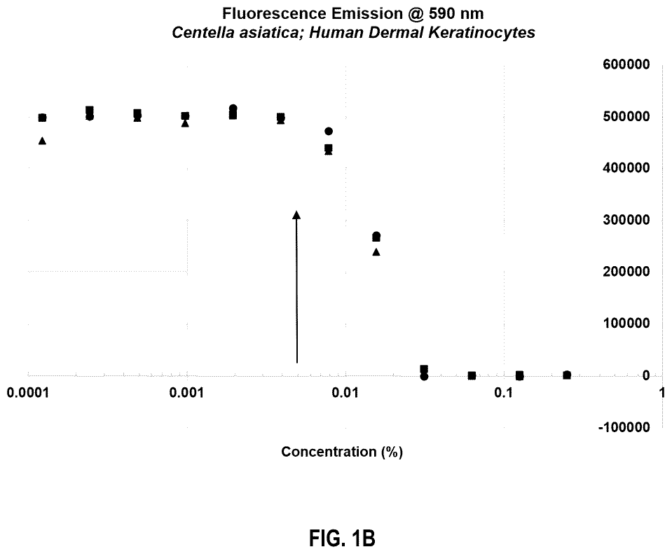

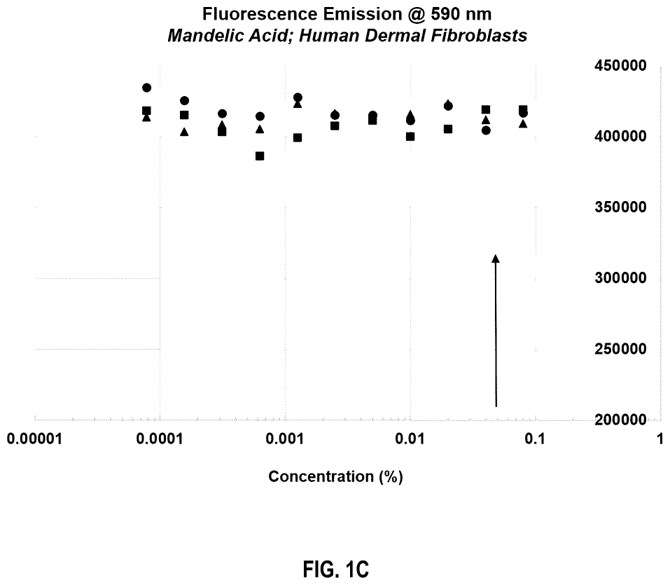

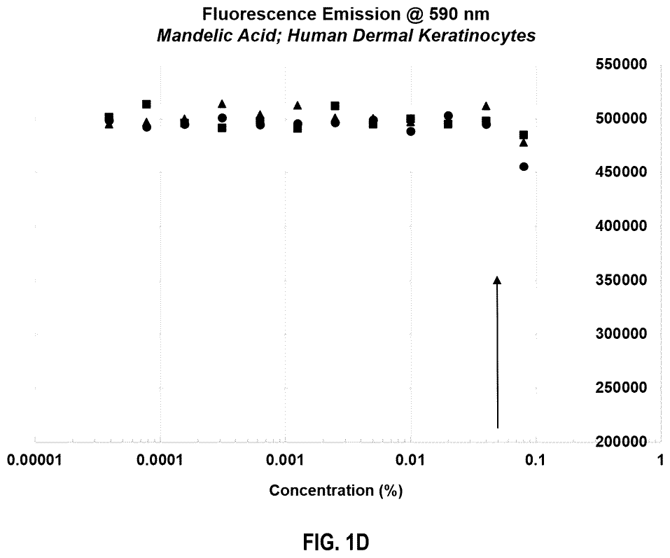

Approximately 10k cells were plated per well in each well of a 96-well plate. A 12-point dilution series of Centella asiatica extract or mandelic acid was performed in triplicate wells. Centella asiatica extract concentrations ranged from 0.00025% to 0.25%, in 2× steps. Mandelic acid concentrations ranged from 0.00004% to 0.08% in 2× steps. After 24 hours of treatment, an alamar blue assay was performed. The plate was then read in an Envision plate reader with 560 nm excitation and 590 nm emission filters to determine cell viability. The fluorescence intensity values were plotted and are shown in FIG. 1A (Centella asiatica, fibroblasts), FIG. 1B (Centella asiatica, keratinocytes), FIG. 1C (mandelic acid, fibroblasts), FIG. 1D (mandelic acid, keratinocytes). The arrows indicate the highest concentrations at which cytotoxicity was not observed in the alamar blue assay, in which the cells are directly exposed to the respective ingredients in the absence of other topical formulation ingredients. Based on the surprising cellular response at the tested concentrations, the present inventors investigated whether negative effects (e.g., inflammation) could be reduced by using Centella asiatica extract and mandelic acid at concentrations lower than those conventionally recommended for their topical use (0.2 to 0.5 wt. % and 0.5 to 6 wt. %, respectively), while also affording a therapeutic effect (e.g., reducing cell senescence, increasing cell turnover, etc.).

### Example 3. In Vitro Analysis of Gene Expression in Human Keratinocytes and Dermal Fibroblasts

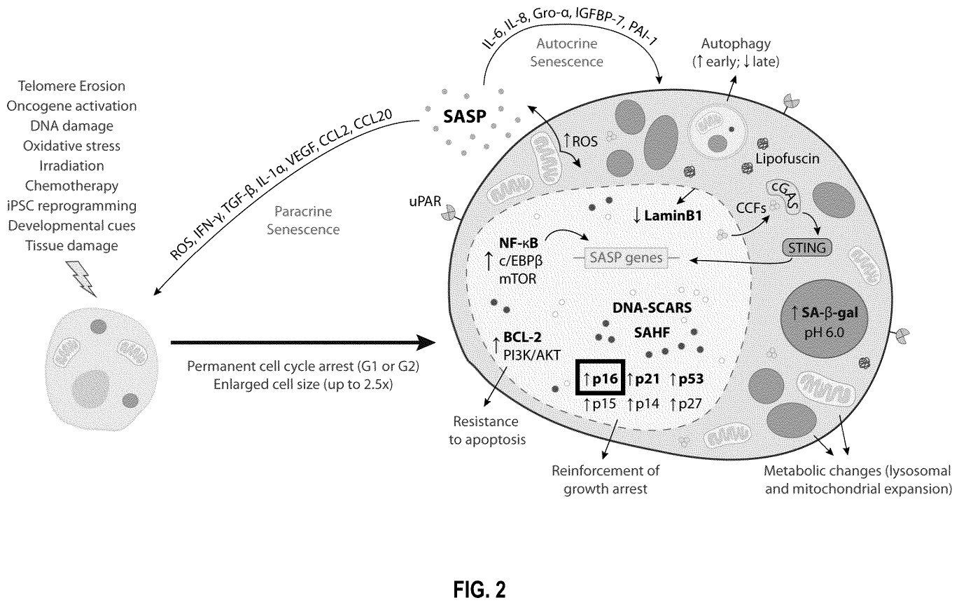

A series of in vitro gene expression analyses were performed to evaluate the effect of topical compositions according to the present disclosure on inflammation, cellular turnover, and senescence. (See FIG. 2, which is a schematic illustration of biomarkers related to inflammation, cellular turnover, and senescence.) Modulation of gene expression in the presence of Centella asiatica abstract, mandelic acid, and a combination of Centella asiatica abstract and mandelic acid were tested to assess effect on genes implicated in the senescence-associated secretory phenotype (SASP), which is a phenotype associated with senescent cells, wherein those cells secrete high levels of inflammatory cytokines, immune modulators, growth factors, and proteases. Human dermal fibroblasts and keratinocytes were grown to confluence and treated with Centella asiatica extract (0.005%), mandelic acid (0.05%), or a combination (same concentrations) of the two actives for 72 hrs. (The selected concentrations of mandelic acid and Centella asiatica extract were determined as the highest concentrations not resulting in cytotoxicity as discussed in Example 2.) RNA was extracted, and RNA sequencing was performed. Differentially expressed genes were determined in comparison to untreated cells. Genes were annotated using the Reactome Database.

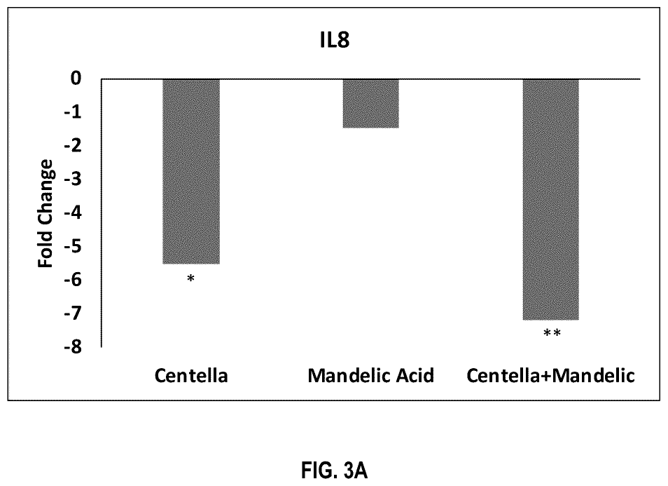

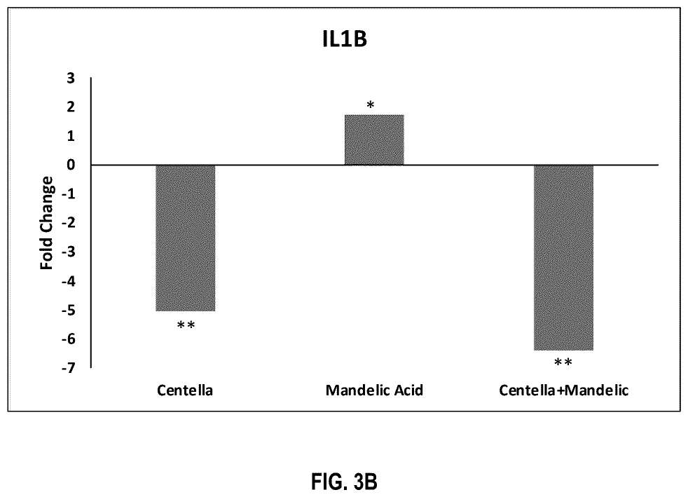

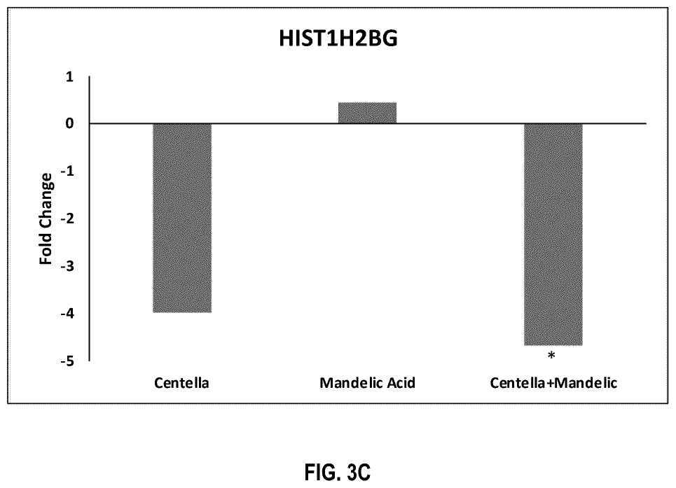

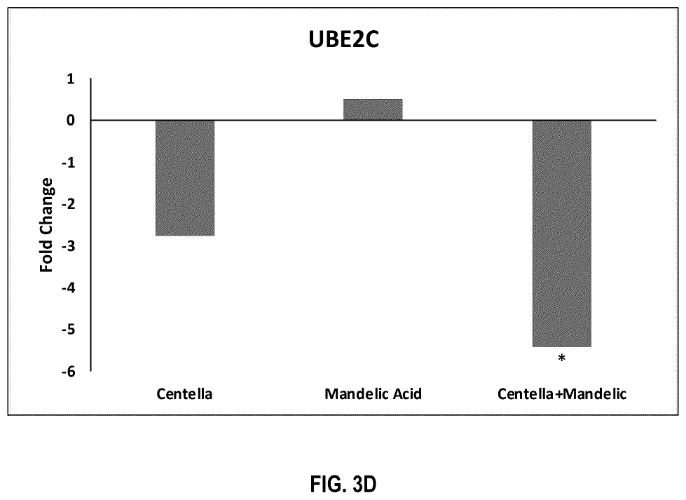

As shown in FIGS. 3A-3D, multiple SASP-related genes which increase inflammation are downregulated by Centella asiatica extract, indicating a protective effect against senescent cells. Moreover, the combination of Centella asiatica extract and mandelic acid synergistically downregulates SASP related genes expressing IL8 (FIG. 3A), IL1B (FIG. 3B), HIST1H2BG (FIG. 3C), and UBE2C (FIG. 3D).

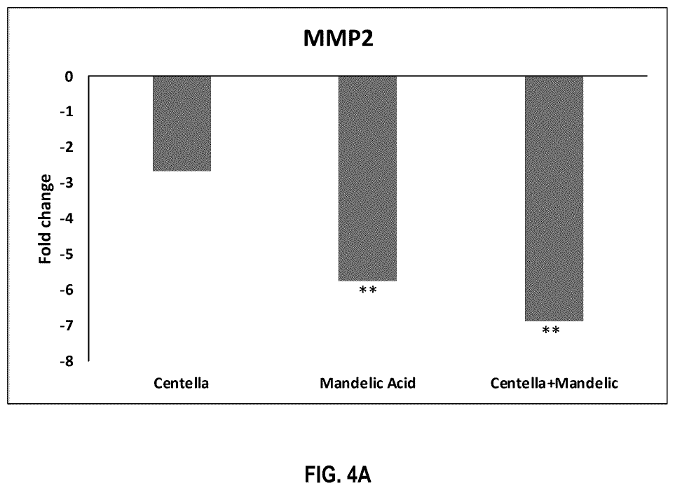

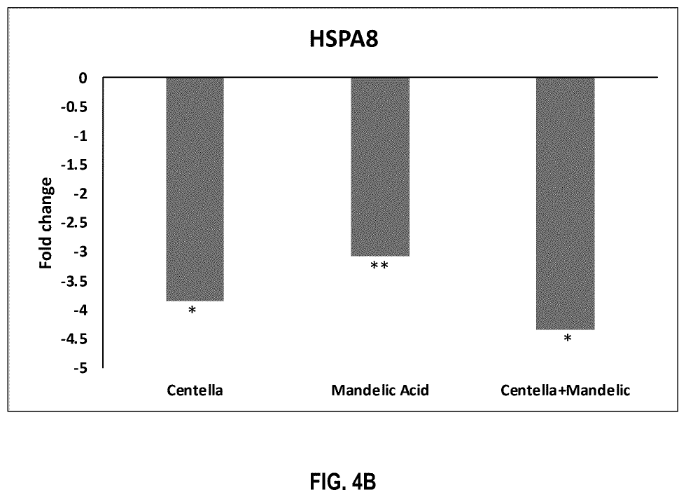

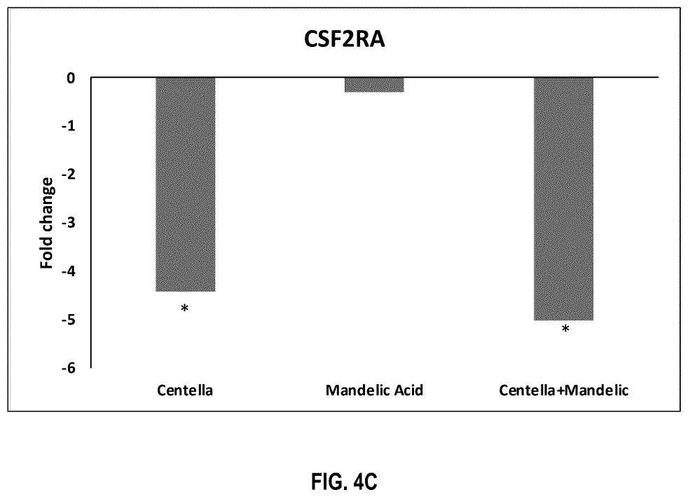

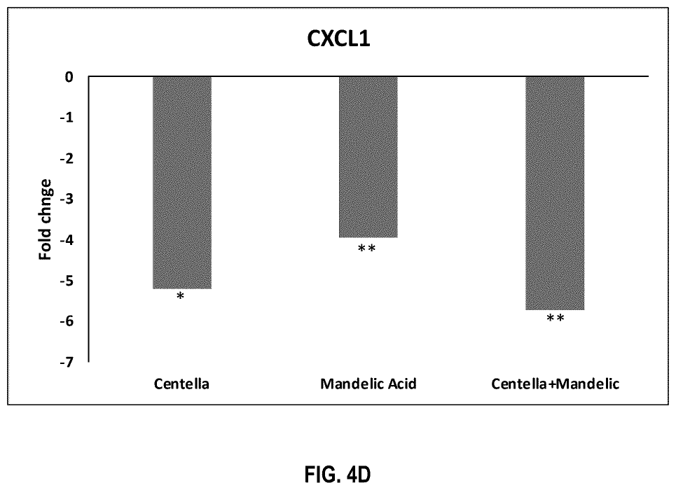

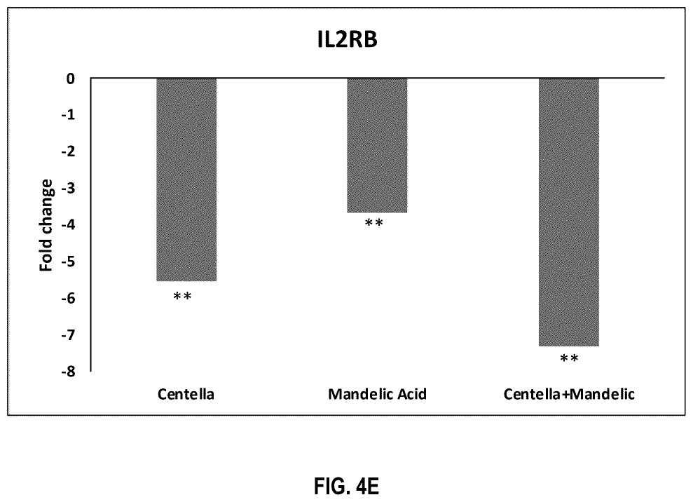

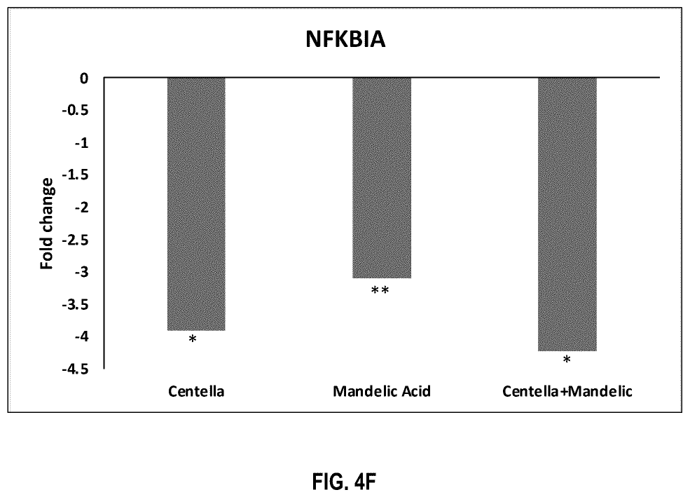

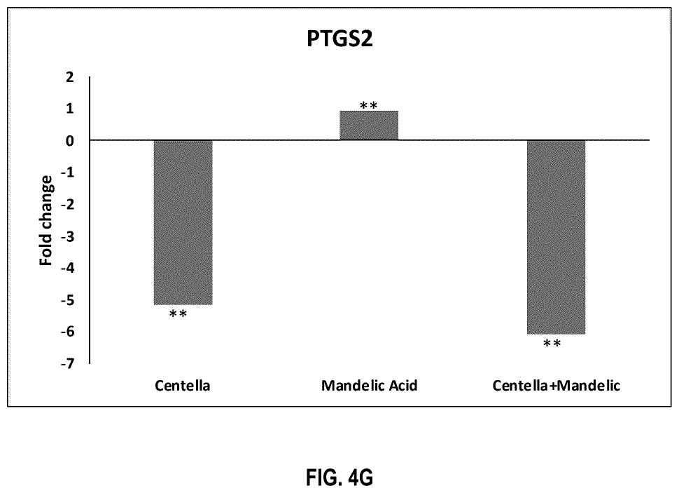

Moreover, as shown in FIGS. 4A-4G, pro-inflammatory gene expression is significantly downregulated by Centella asiatica extract and by mandelic acid, individually and in combination, in particular for MMP2 (FIG. 4A), HSPA8 (FIG. 4B), CSF2RA (FIG. 4C), CXCL1 (FIG. 4D), IL2RB (FIG. 4E), NFKBIA (FIG. 4F), and PTG52 (FIG. 4G). Moreover, the combination of Centella asiatica extract and mandelic acid synergistically downregulates expression of genes related to general inflammation, which may be related to the downregulation of SASP-related genes. These data are summarized in Table 2.

TABLE 2
Modulation of Gene Expression (Fold Change) by Centella Asiatica
Extract, Mandelic Acid, and Combination of Centella Asiatica
Extract and Mandelic Acid

| Marker | Centella | Mandelic Acid | Centella + Mandelic |
|---|---|---|---|
| IL8 | −5.50 | −1.46 | −7.17 |
| IL1B | −5.05 | 1.73 | −6.41 |
| TNF | −8.62 | −2.69 | −7.83 |
| HIST1H4H | −4.82 | −0.54 | −4.16 |
| HIST1H2BG | −3.98 | 0.45 | −4.68 |
| UBE2C | −2.77 | 0.51 | −5.44 |
| UBB | −5.06 | −3.87 | −5.69 |
| UBC | −3.43 | −1.09 | −3.16 |
| CEBPB | −4.63 | −2.45 | −3.69 |
| HIST1H2BD | −4.05 | 0.55 | −4.02 |
| HIST1H4E | −11.59 | 0.60 | −6.62 |
| HIST1H3D | −11.21 | −0.23 | −11.00 |

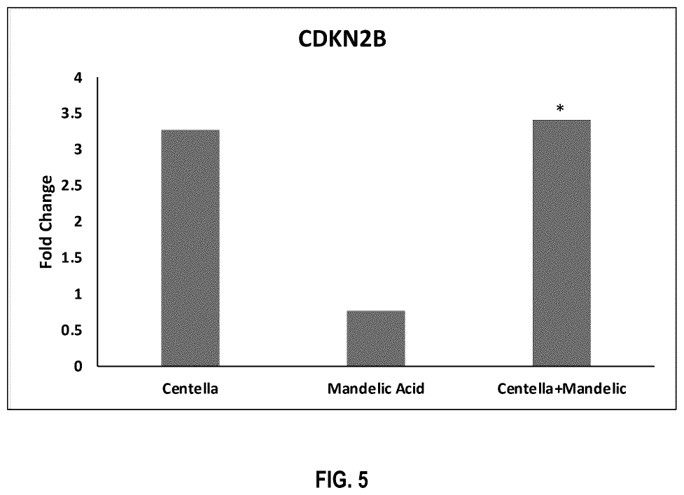

As shown in FIG. 5, the Centella asiatica extract and mandelic acid, individually and in combination, upregulate expression of p15 (CDKN2B), which is implicated in evasion of oxidative stress-induced senescence due to defective p16INK4A binding to CDK4. Thus, treatment with compositions according to the present disclosure could protect against cell senescence in keratinocytes and decrease the presence of senescent cells in the dermis.

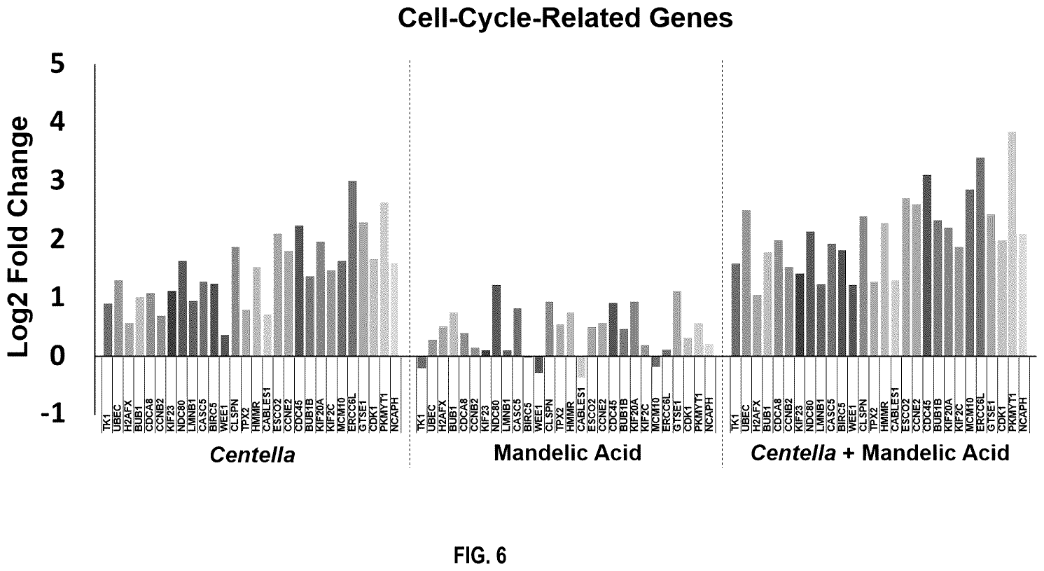

Referring now to FIG. 6, the combination of Centella asiatica extract and mandelic acid synergistically and significantly upregulates the expression of cell cycle-related genes TK1, UBEC, H2AFX, BUB1, CDCA8, CCNB2, KIF23, NDC80, LMNB1, CASC5, BIRC5, WEE1, CLSPN, TPX2, HMMR, CABLES1, ESCO2, CCNE2, CDC45, BUB1B, KIF20A, KIF2C, MCM10, ERCC6L, GTSE1, CDK1, PKMYT1, and NCAPH in dermal fibroblasts. Remarkably, only the combination of Centella asiatica extract and mandelic acid significantly upregulates all the genes shown (y-axis shows log 2 fold change). The data indicates improved cell turnover and thus, reduced cellular senescence.

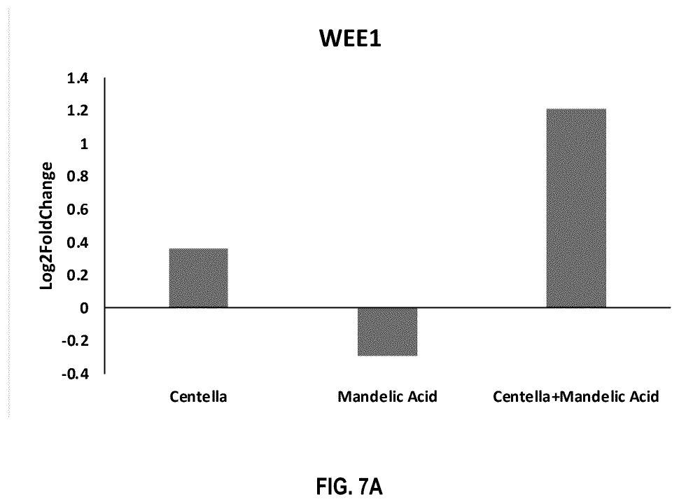

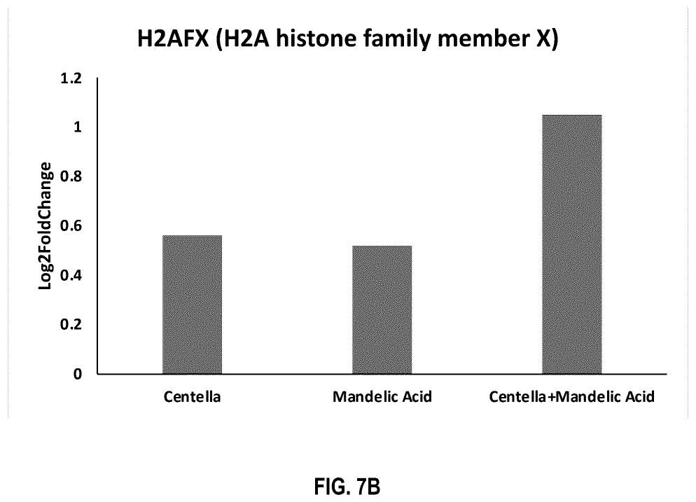

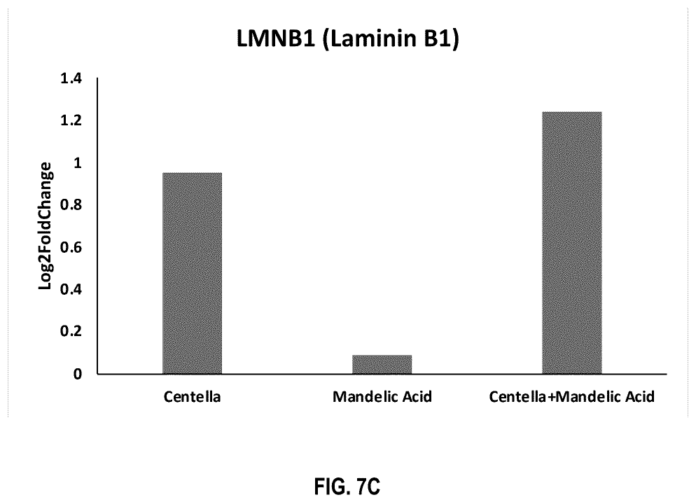

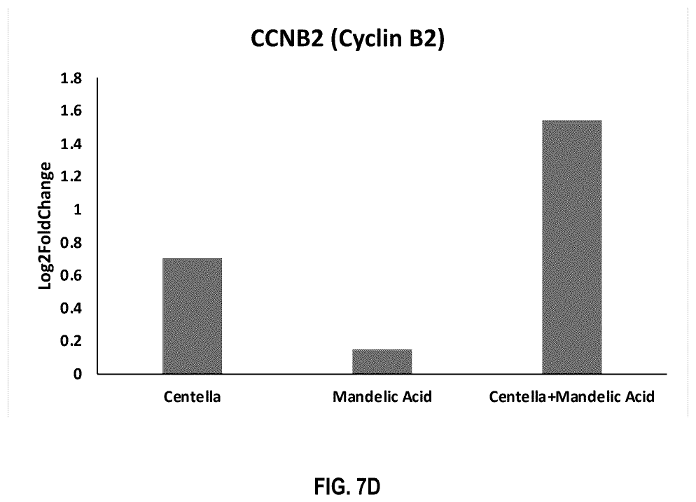

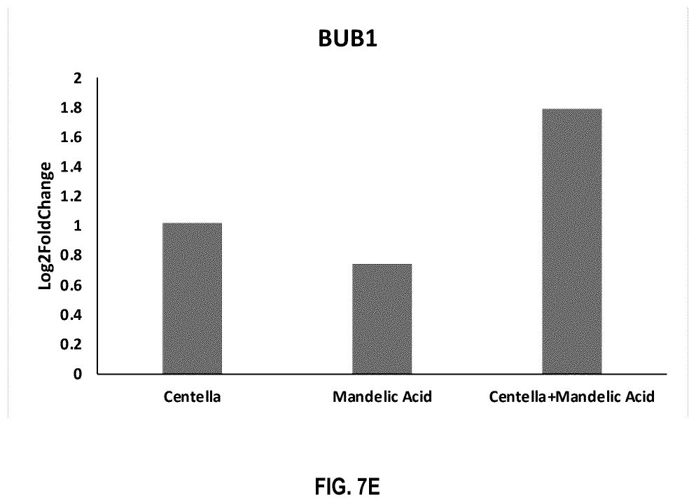

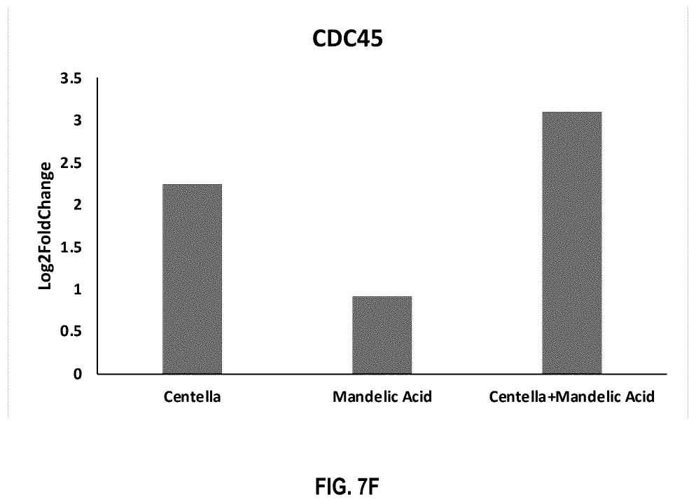

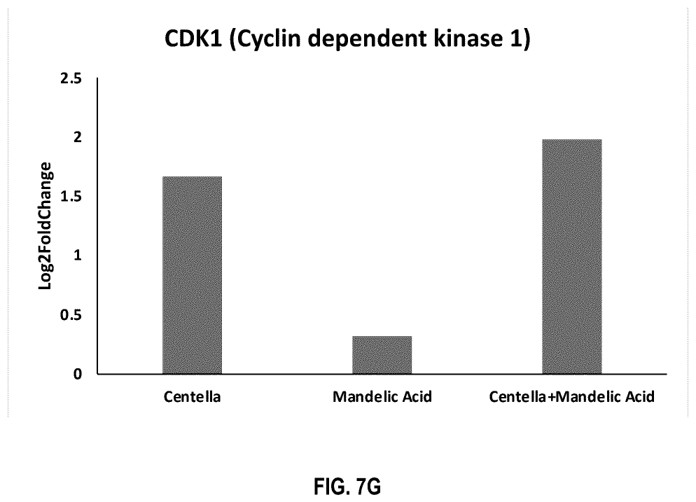

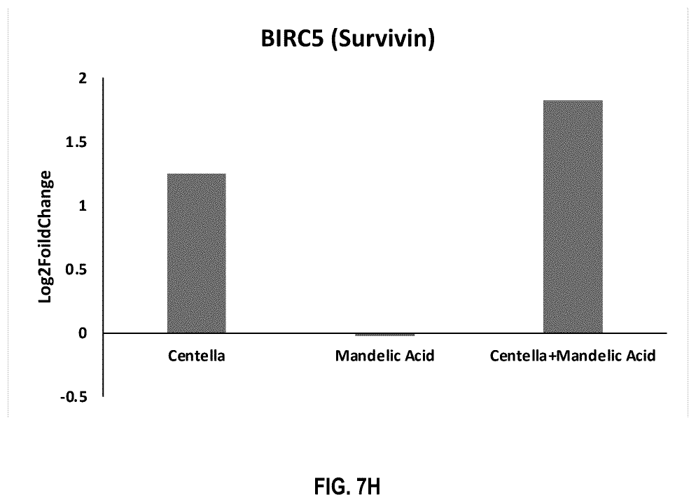

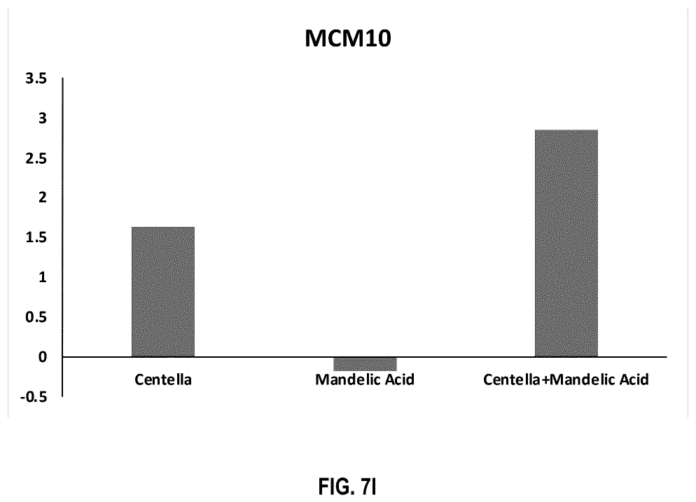

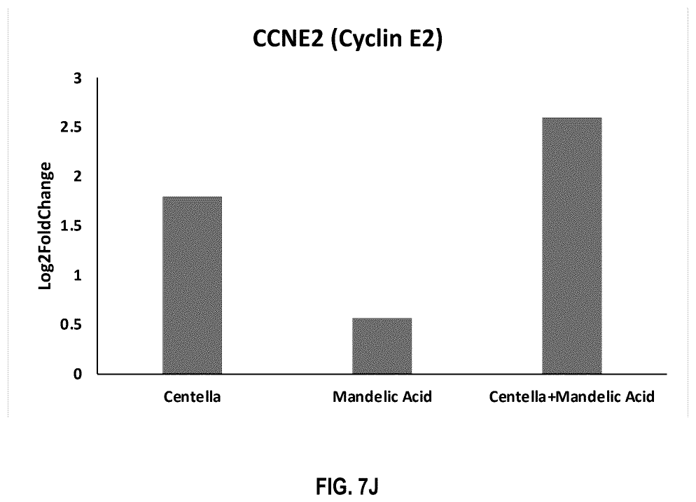

Additionally, referring now to FIGS. 7A-7J, the combination of Centella asiatica extract and mandelic acid synergistically upregulates the expression of p16-interacting genes WEE1 (FIG. 7A), H2AFX (FIG. 7B), LMNB1 (FIG. 7C), CCNB2 (FIG. 7D), BUB1 (FIG. 7E), CDC45 (FIG. 7F), CDK1 (FIG. 7G), BIRC5 (FIG. 7H), MCM10 (FIG. 7I), and CCNE2 (FIG. 7J), indicating that the combination counteracts p16 in fibroblasts, reversing cellular senescence.

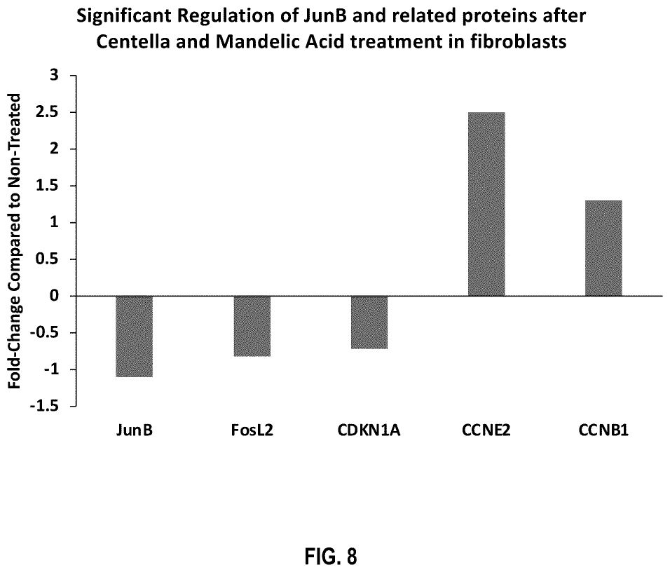

Dermatoporosis is associated with a loss of ECM (collagen, elastin, GAGs etc) causing thinning of the skin, fragility, and vessel leakage. This breakdown is linked to ROS generation which stimulates the activation of transcription factors (such as AP-1) and the synthesis of matrix metalloproteinases (MMPs). JunB is a major component of transcription factor AP-1. Thus, downregulating this factor will result in less or limitation of ECM breakdown. Referring now to FIG. 8, fibroblasts treated with a combination of Centella asiatica extract (0.005%) and mandelic acid (0.05%) show significant downregulation of JunB and FosL2, both of which are transcription factors and members of the AP-1 complex. This downregulation blocks the ability of p21 (CDKN1A) to inhibit the cell cycle and in turn, activates cell cycle progression as shown by the upregulation of CyclinE2 (CCNE2) and CyclinB Id (CCNB1). (All fold changes were significant, with p-values <0.05.)

Taken together, the gene expression data show that the combination of Centella asiatica extract and mandelic acid induce a robust and synergistic effect in: (i) significantly decreasing the inflammatory secretome (SASP) of senescent cells, thus evading senescent cell activity in keratinocytes; (ii) increasing dermal fibroblast cell turnover; and (iii) diminishing p16 (senescent cell marker, cell cycle blocker), indicating a decreased cellular senescence overall in fibroblasts.

### Example 4. Ex Vivo Analysis of Cell Senescent Activity

To investigate the effect of topical compositions according to the present disclosure on senescent cell response when topically applied to skin, a test composition was prepared according to Example 1 and was tested ex vivo on discarded human skin samples. Biopsies were made from discarded human skin samples, to place tissue into 24-well plates for culture. After acclimation, the skin samples were treated with the test composition once daily for 10 days. Specimens were stained with p16 to assess senescent activity, using untreated skin samples as a baseline.

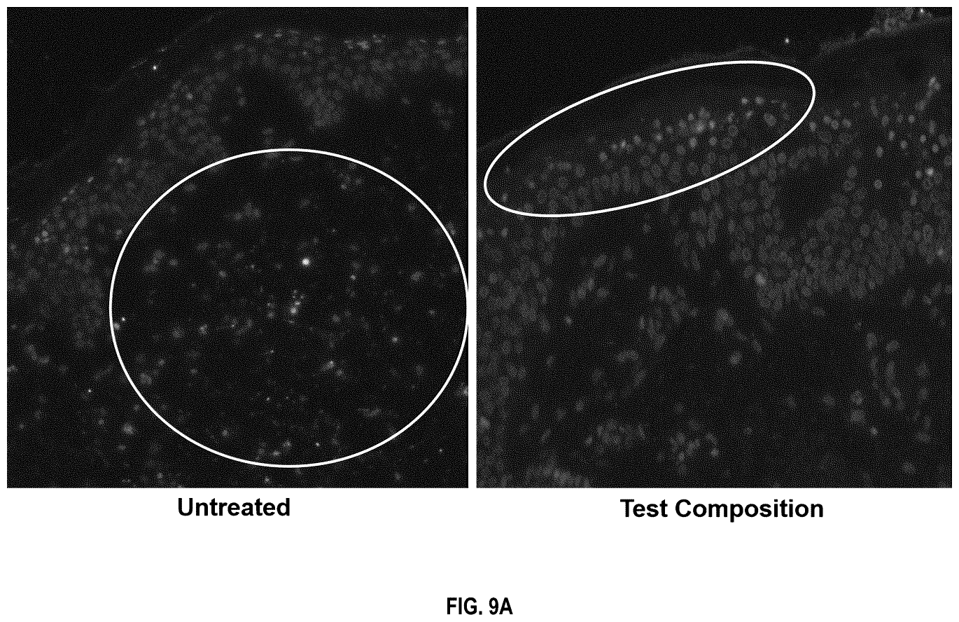

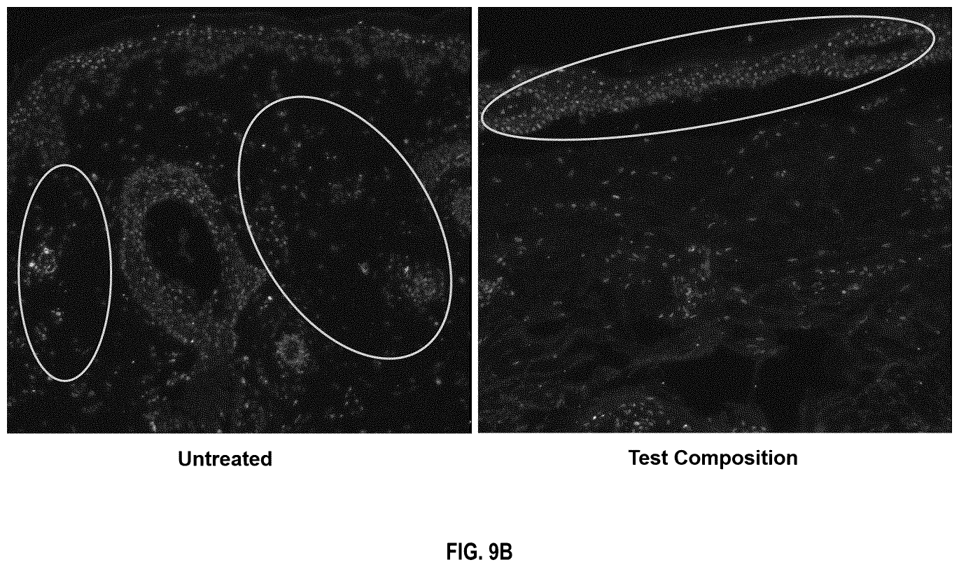

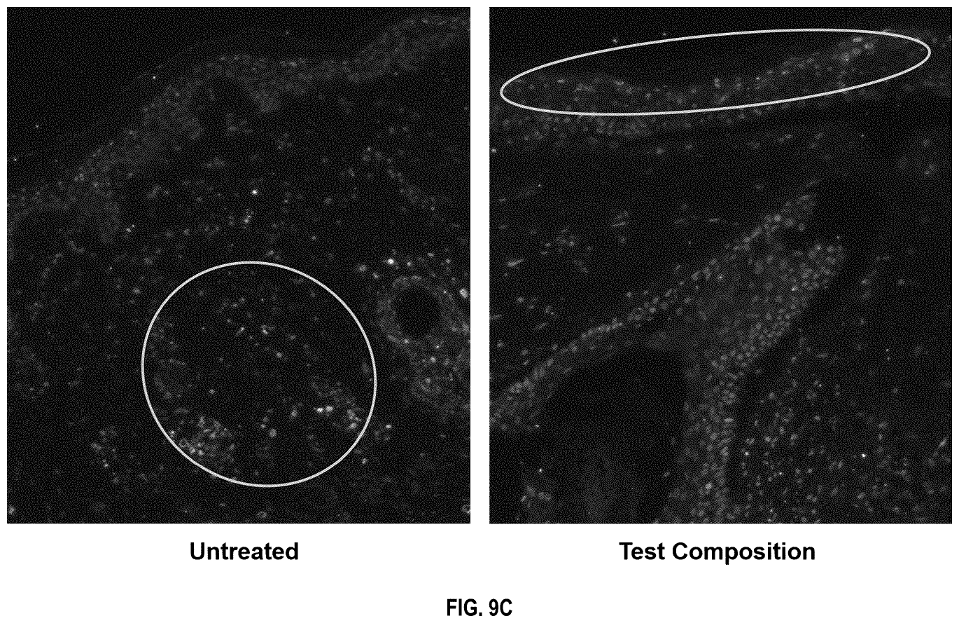

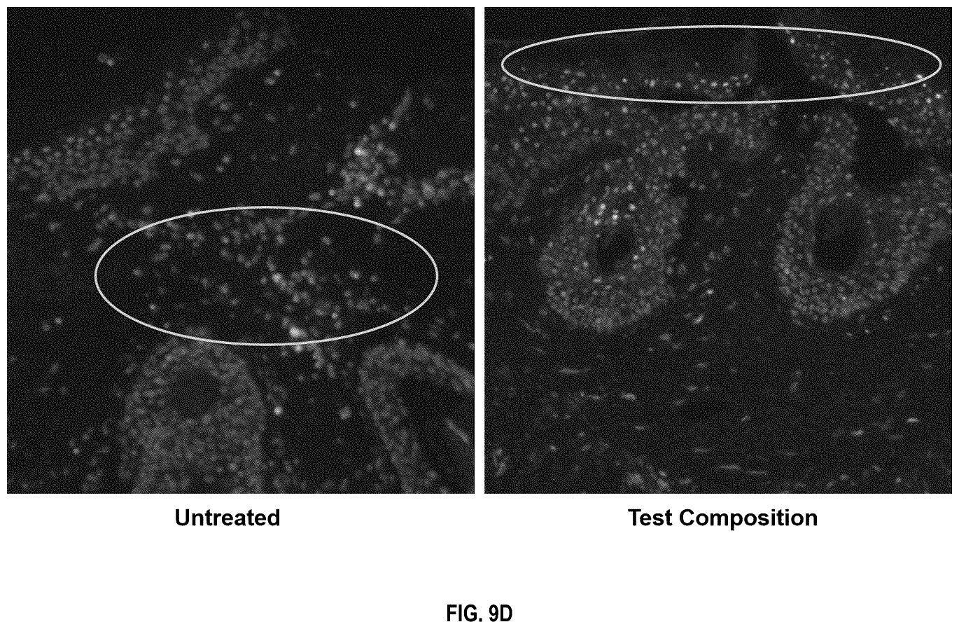

FIGS. 9A-9D show images of p16-stained skin samples for untreated skin (left) versus skin treated using the test composition (right). Cell nuclei appear in blue, while p16 appears in yellow. The untreated skin shows evidence of p16 (senescent cells) in the dermis. In contrast, the samples treated with the test composition show loss of senescent cells in the dermis and evidence of senescent cells in the epidermis. In several of the images, high concentrations of senescent cells are observed in hair follicles, where cells undergo natural senescence as part of the hair cycle. These senescent cells are expected to be present and are not considered in this analysis.

The ex vivo results corroborate the gene expression data (Example 2) related to p16 senescent activity, as the test composition comprising Centella asiatica extract and mandelic acid appears to decrease and even counteract p16 activity. The ex vivo results also indicate increased dermal cellular turnover, evidenced by decreased senescent cell population in the dermis after treatment, compared to untreated skin.

Moreover, the compositions according to the present disclosure surprisingly appear to promote clearance of dermal senescent cells out of the dermis and into the epidermis. Without being bound to any particular theory, the test composition appears to potentially promote clearance of senescent cells through a signaling mechanism that initiates in the epidermis, signaling down to dermal senescent cells (possibly due to the collagen-stimulating effect of Centella asiatica extract), reversing senescence in dermis and then promoting epidermal turnover with increased epidermal senescent cells (and protection from these cells) and terminally differentiated cells appearing in upper epidermis. This suggests a form of senescent cell clearance and cellular revival, which would be highly advantageous for management of dermatoporosis.

While the foregoing terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

In addition, where features or aspects of the disclosure are described in terms of Markush groups, those skilled in the art will recognize that the disclosure is also thereby described in terms of any individual member or subgroup of members of the Markush group.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and equivalents thereof.

All patents, patent applications, provisional applications, and publications referred to or cited herein are incorporated by reference in their entirety, including all figures and tables, to the extent they are not inconsistent with the explicit teachings of this specification.

Reference will now be made in detail to some specific embodiments contemplated by the present disclosure. While various embodiments are described herein, it will be understood that it is not intended to limit the present technology to the described embodiments. On the contrary, it is intended to cover alternatives, modifications, and equivalents as may be included within the spirit and scope of the technology as defined by the appended claims.

What is claimed is:

1. A topical composition, comprising:
Centella asiatica extract encapsulated within liposomes wherein the Centella asiatica extract is present at a concentration of at least about 0.01 wt. % and less than 0.2 wt. %, relative to the total weight of the composition; and
mandelic acid present at a concentration of at least about 0.01 wt. % and less than 0.5 wt. %, relative to the total weight of the composition,
wherein the composition has a pH of less than 5.

2. The topical composition of claim 1, wherein the composition is in the form of an emulsion.

3. The topical composition of claim 1, wherein the pH of the composition is greater than or equal to 4 and less than 5.

4. The topical composition of claim 1, wherein the Centella asiatica extract is present at a concentration of about 0.01 wt. % to about 0.1 wt. %, relative to the total weight of the composition.

5. The topical composition of claim 1, wherein the Centella asiatica extract comprises triterpenes.

6. The topical composition of claim 5, wherein the triterpenes are one or more selected from the group consisting of: madecassoside, asiaticoside, madecassic acid, and asiatic acid.

7. The topical composition of claim 5, wherein the triterpenes are present in the Centella asiatica extract at a concentration of at least about 0.005 wt. % and less than 0.2 wt. %, relative to the total weight of the composition.

8. The topical composition of claim 1, wherein the liposomes comprise lecithin, propanediol, or a combination thereof.

9. The topical composition of claim 1, wherein the liposomes comprise an antioxidant.

10. The topical composition of claim 9, wherein the antioxidant is tocopherol, tocopheryl acetate, or a combination thereof.

11. The topical composition of claim 1, wherein the liposomes have a mean particle size of 100 nm to 300 nm.

12. The topical composition of claim 1, wherein the mandelic acid is present at a concentration of about 0.05 wt. % to about 0.3 wt. %, relative to the total weight of the composition.

13. The topical composition of claim 1, wherein the Centella asiatica extract and the mandelic acid are present in a weight ratio of about 1:5 to 1:15.

14. The topical composition of claim 1, further comprising an antioxidant separate from any antioxidant in the liposomes.

15. The topical composition of claim 1, further comprising glycerin.

16. The topical composition of claim 1, wherein the composition is effective to:
(i) reduce the number of senescent cells in mammalian dermis;
(ii) reduce the senescence-associated secretory phenotype (SASP) in mammalian epidermal cells;
(iii) downregulate at least one SASP-associated gene selected from the group consisting of IL8, IL1B, HIST1H2BG, and UBE2C;
(iv) upregulate expression of at least one cell cycle gene in mammalian dermal fibroblasts, wherein the at least one cell cycle gene in mammalian fibroblasts is selected from the group consisting of: TK1, NDC80, HMMR, KIF2C, UBEC, LMNB1, CABLES1, MCM10, H2AFX, CASC5, ESCO2, ERCC6L, BUB1, BIRC5, CCNE2, GTSE1, CDCA8, WEE1, CDC45, CDK1, CCNB2, CLSPN, BUB1B, PKMYT1, KIF23, TPX2, KIF20A, and NCAPH;
(v) downregulate at least one gene associated with skin inflammation selected from the group consisting of: MMP2, HSPA8, CSF2RA, CXCL1, IL2RB, NFKBIA, and PTGS2;
(vi) downregulate a transcription factor of the AP-1 complex; or
(vii) activate cell cycle progression by upregulating CCNE2, CCNB1, or a combination thereof.

17. A topical composition, comprising:
an alpha hydroxy acid (AHA); and
triterpenes selected from the group consisting of: madecassoside, asiaticoside, madecassic acid, and asiatic acid,
wherein the composition has a pH of 4 to 5.

18. The topical composition of claim 17, wherein the triterpenes are encapsulated within a liposome.

19. The topical composition of claim 17, wherein the AHA comprises mandelic acid.

20. A method for treating skin affected by dermatoporosis, increasing fibroblast cell turnover in mammalian skin, reducing the number of senescent cells in mammalian dermis, reducing the senescence-associated secretory phenotype (SASP) in mammalian epidermal cells, increasing the expression of at least one cell cycle gene in mammalian dermal fibroblasts downregulating at least one SASP-associated gene in mammalian skin, downregulating at least one gene associated with skin inflammation, downregulating at least one transcription factor of the AP-1 complex, or activating cell cycle progression, the method comprising:
administering the composition according to claim 1 to skin.
